(19) United States
(12) Patent Application Publication
(10) Pub. No.: US 2020/0160985 A1
(43) Pub. Date: May 21, 2020

(54) Patient-Centric Eco-System with Automated Workflow and Facility Manager for Improved Delivery of Medical Services

(71) Applicant: Symplast Acquisition Inc., Plantation, FL (US)

(72) Inventors: Shashi Kusuma, Plantation, FL (US); Vadim Koystinen, Vienna, VA (US); Facundo Formica, Fort Lauderdale, FL (US)

(21) Appl. No.: 16/689,773

(22) Filed: Nov. 20, 2019

Related U.S. Application Data
(60) Provisional application No. 62/769,797, filed on Nov. 20, 2018.
(60) Provisional application No. 62/769,788, filed on Nov. 20, 2018.

(51) Int. Cl. G16H 40/20; G16H 30/00; G16H 10/60; G16H 80/00; G06Q 10/06

(52) U.S. Cl. CPC G16H 40/20; G16H 30/00; G06Q 10/06393; G16H 80/00; G16H 10/60

(57) ABSTRACT

The patient-centric eco-system operates in healthcare facility's (HC-FAC) treatment/recovery/waiting rooms using audio/visual (presence) sensors and image sensors (serial static images or video). Sensors capture patient presence sensory data in rooms. Computing devices process data and obtain room-to-room patient unique transition data, patient unique treatment/recovery timing data, and treatment/recovery image data. System digitally tags and segments the treatment/recovery images (serial static images or video) to generate time-stamped patient unique treatment/recovery image data which is serial static images or video clip(s). Patient/provider replay command displays processed images or video clip. The display may be on a multimedia medical documentation presentation platform. To monitor and track patient flow through HC-FAC, a basic process flow user interface (UI) is a 3-column display (arrivals, treatment/recovery and check-out) tracking all patients at the HC-FAC with patient-display tiles manual/auto moved column-to-column as each patient transitions through HC-FAC.

This is a regular, non-provisional patent application based upon and claiming the benefit of provisional patent application Ser. No. 62/769,797, filed Nov. 20, 2018, now pending, entitled “Patient-Centric Eco-System with Automated Workflow and Facility Manager for Improved Delivery of Medical Services” and also this non-provisional patent application is based upon and claiming the benefit of provisional patent application Ser. No. 62/769,788, filed Nov. 20, 2018, now pending, entitled “Multimedia Medical Documentation Process and Presentation Platform” and the contents of serial nos. 62/769,797 and 62/769,788 is incorporated herein by reference thereto.

### BACKGROUND OF THE INVENTION

Currently, Healthcare (HC) systems are not dynamically configured to adjust HC time and HC Facility (“FAC”) room, patient flow, or load utilization. As for HC-FAC spaces, waiting areas, medical preparation rooms or spaces, examination rooms, treatment rooms, post-op rooms and spaces and check-out or payment spaces, these are rarely used efficiently given typical patient loads and different classes of patients.

There is a need to re-configure a physician's display monitor to call up and study different digital media files captured during the patient's interaction with the HC-FAC. With the advent of relatively inexpensive data acquisition devices (video, still image cameras, portable phones with cameras and sound recording features, etc.), cheap data storage, and fast network communications, the physician should be able to access, from a single main screen these videos, images, electronically stored information (ESI), medical data, and textual material derived from audio tracks, all shown on an easily comprehensible display, with labels and time based presentations.

### OBJECTS OF THE INVENTION

It is an object of the present invention to assist the scheduling of patient appointments and to balance resources such as medical healthcare (“HC”) personnel, HC facility rooms within the medical practice building, and medical equipment.

It is another object of the present invention to increase efficiency of the HC practice and enhance the patient's experience because common HC prior art calendaring and scheduling displays do not provide the whole picture of the HC practice.

It is a further object of the present invention to present a holistic view of the current day at the HC practice and focus on what is happening now versus merely showing a day view of timeslots.

It is an additional object of the present invention to present to the HC physician and HC staff a practice flow user interface (UI) that provides the HC practitioner and staff with an overall understanding of the patients at that office and the patients' current needs at the HD-FAC.

It as another object of the present invention to provide a system and method that uses a software platform with a comprehensive, HC-FAC wide data acquisition, with automatic tagging of patient-driven events. Artificial Intelligence (AI) curation, to create a paradigm shift in the delivery of medical services throughout the FAC with a number of algorithms to increase (a) spatial (physical) efficiency, (b) personnel, staffing and FAC scheduling efficiencies, (c) time based services, and (d) product utilization efficiency.

It is another object of the present invention to provide a process flow UI view which graphically illustrates the current demands on the HC practice and HC staff, and also enables efficient use of time and resources to ensure an excellent experience for the patients entrusting their care to the practice that day. This feature is not intended to replace the typical calendar for scheduling appointments, but it will provide a much more holistic representation of the current day at the practice.

It is another object of the present invention to provide multimedia medical documentation process and presentation platform (a story board) which combines, in a singular display panel (sometimes called a dashboard display), multimedia documents such as video clips or video data blocks, digital data streams, audio/voice data, image data, and textual electronic stored information (ESI) which is input and stored in the Patient flow process.

### SUMMARY OF THE INVENTION

The invention is designed as a KANBAN style, LEAN, efficient patient flow process. Lean processing techniques seek to eliminate waste from the medical delivery service process resulting in improved efficiency, effectiveness, and profitability. The system and method combines modern immediate actionable scheduling with patient education methodologies with educational displays and for cross-reference marketing and upsell activities. Also, the system and method provides a room management planner and an actionable healthcare provider-patient scheduling system coupled to an artificial intelligence (AI) engine to process the facility's operational aspects. The AI outputs drive all of these functions such as suggesting better staff scheduling, adjusting room utilization and increasing patient throughput. The system and method includes processes for scheduling the patient to a room, and the sequential activation of different levels of HC providers (e.g., a medical technician to draw blood, a nurse to prepare the patient for the surgical operation, and the doctor to perform the surgery, and then a nurse for post-op care and treatment) to the designated room. After the post-op or recovery treatment, the patient is directed to the checkout stations at the facility. Further follow-ups are provided to the patient per the scheduling system via the patient's cell phone or smart phone and scheduling prompts generated by the system and method.

The system and method also learns the general timelines for the patient, either as a class of patients or as individual patients, who repeatedly interact with the system and method. For example, some patients as a class (for example, cancer patients) need more time with HC oncology specialists as compared with patients having knee injuries. These are treatment classes which are identified in a taxonomic system wherein AI is used and the AI outputs are curated by the system operator (Sys Op) to provide better patient care and improved staff and facility (FAC) and FAC room utilization. Some patients can be grouped by age brackets (age bracket classes suggested by the taxonomic AI system). The different patient classes may be also organized by type of employment (office workers vs. blue collar factory workers vs. construction workers).

The system and method uses a software platform with a comprehensive, HC FAC-wide data acquisition, with automatic tagging of patient-driven events, AI curation guided by an HC provider or Sys Op, to create a paradigm shift in the delivery of medical services over the FAC. Algorithms increase (a) spatial (physical) efficiency, (b) personnel, staffing and FAC scheduling efficiency, © time-based services delivery and (d) product utilization efficiency.

For example, the system and method may start with an initial program setting of how long a person wishing a certain Botox treatment (e.g., a Botox forehead treatment) spends in the FAC med prep room or space, the exam room, the treatment room, and the post-op room. The system also can be pre-programmed to staff that room with designated HC professionals at certain times for patient Alpha, herein “Pat Alpha,” “Pat Beta”, etc.

The intelligent system and method captures voice and video data for Pat Alpha during first, second and possibly third forehead Botox treatments spread over a six week period (a super set of treatments). The system and method captures voice and video data in each of the HC-FAC physical plant areas (the med prep room or space, the exam room, the treatment room, and the post-op room). The system notes when Pat Alpha arrives in the waiting room (because his or her entrance is logged into the system) and automatically determines using the super set treatment data that the initial program settings (patient time in room, staff time in room) are “too-long.” Pat Alpha moves through the HD-FAC because he or she is being treated during the lunch hour.

The AI-driven system then shortens the scheduling program patient time for Pat Alpha in the HC-FAC rooms, and tightens the HC staff interaction and time for Pat Alpha in each room, therefore freeing up the HC-FAC rooms and HC staff for other patients. This increases revenue to the HC-FAC, and decreases staff time (also results in an increase in revenue), and better serves Pat Alpha due to the limited lunch time period.

Further, the system notes that Patient Pat Alpha and patients O, P, and Q, all on different days over different months (a super set of multiple patient treatments), undergo forehead Botox treatments between the times of 11 AM and 2 PM. The AI-driven system and method automatically classifies these O, P, Q patients as “quick patients”. Since the system and method identifies this M-F, 11 AM and 2 PM, Botox “forehead” trend, quick patient class, the HC-FAC can reduce the fees charged to Patients Alpha, O, P, and Q, because more 11 AM-2 PM patients can be treated at relatively the same price point and generate sufficient revenues for the HC-FAC within a time-based revenue period. This pricing plan also increases HC patient volume if the fee reduction is marketed to existing and new, prospective patients.

The comprehensive data acquisition, associated with the AI scheduler, first gathers data, identifies data trends, and then, after curation by an HC professional, recommends a greet, med prep, pre-op, treatment, and post-op/recovery schedule. The AI scheduler merges physical spaces and staff time.

The AI scheduler learns the different timelines that different providers have for different patients (and classes of patients). Different HC physicians have different “patient face time” periods. The AI scheduler will create different algorithms and different paradigms for different classes of patients, different treatments, matching different HC staffers having different skill sets with patients. As an example, the initial setting for Doctor G has set a 20 minute scheduled time for Treatment K. Over the past 30 days, the AI scheduler learns that Doctor G takes 30 minutes for this Treatment K for Patient Class x,y. So now the AI scheduler recommends that Doctor G should allocate 30 minutes for Treatment K for Patient Class x,y. The curation involves Doctor G confirming the longer treatment periods for Patient Class x,y. Of course, the System Operator (Sys Op) can curate time-in-room and staffing requirements for treatments q, r, s and t and confirm patient classes x, y and z, with different scheduling for spatial, staff and room occupancy times. Some rooms have different equipment and the use of this different equipment generates different revenues for the HC-FAC. “Equipment usage” is also a data acquisition event, subject to AI scheduling. Equipment ON/OFF times can be captured by sensors and processed by the AI scheduler.

Sometimes it is the patient, as an individual (not as a class), that needs more time and assistance. Assuming the patient repetitively visits the HC-FAC, the AI scheduler learns the patient's routine, and, for example, learns that patient Delta, herein “Pat Delta,” needs more staff service or more care. For Pat Delta, the AI scheduler identifies the HC-FAC room space times and designated skilled staff (maybe a recovery room used after Pat Delta leaves the post-op room) and therefore can track and create added value for Pat Delta who needs more time for the treatment. This patient-centric treatment regime, unique to Pat Delta, enhances patient outcomes and creates good will towards the HC provider. This good normally increases referrals to the provider.

This patient-customization feature by the AI scheduler can recommend a different price schedule for Patient Delta. Further and just as important, Patient Delta's recovery after she leaves the HC-FAC may be quicker, with higher drug or post-op compliance levels, if she is given more time in the designated HC room with appropriately trained HC staff

With the AI scheduler, the system and method collects data, analyzes the time/room/staffing per treatment utilization with the initial time/room/staffing per treatment, recommends changes to time/room/staffing per treatment, permits customization on a Per-patient level, and then permits the Sys Op or Administrator to adjust the compensation schedules and the fee schedules to achieve better and more efficient utilization of physical space, staff, equipment usage and product usage over the wider range of patients using the HC-FAC and the professional services.

The AI Scheduler automatically adjusts for patient group or classes, adjusts for individual repetitive patient needs, and ties that intelligent process to control medical facility utilization and staffing. With this system efficiency, the Administrator can properly configure pricing within the practice to drive value and decrease cost and be more efficient.

In conjunction with the comprehensive data acquisition, a multimedia medical documentation process and presentation platform is configured as a storyboard or dashboard panel showing video blocks or data streams, audio/voice data, image data, and electronic text and ESI gathered during the patient flow process. Essentially, the patient flow information system operates as a database that stores patient data, healthcare (HC) worker user data, and HC facility data. This database also includes stored video blocks or data streams, audio/voice data, image data, and electronic ESI text which includes both (i) user generate text input and (ii) AI generated digitally formatted text, herein E-TXT. The database includes labels, tags and Text strings (TXT-Str) for the curated AV/Img data streams. All this data (AV, Img, E-TXT) is time stamped, labeled and tagged. The added and displayed text-strings or TXT-Str data is displayed beneath each AV/Img displayed data. The AV/Img and textual data is displayed on a multimedia storyboard which includes video, audio, textual material, image material as well as a timeline bar. This single format display, with a timeline bar, enables the physician to quickly pull up key reports, images, and video presented as a patient unique diagnosis and treatment dashboard.

In summary, the basic patient-centric eco-system with an automated workflow and facility manager for improved delivery of medical services is a computer-based method for data processing in a healthcare facility having a plurality of treatment rooms, a recovery room and a waiting room. Each room has an audio sensor generating audio data for voice recognition and/or an image sensor generating image data. This image data consists of a series of static images or video. The audio sensors and image sensors generate presence sensory data for the patients in the respective rooms. Each treatment room has one or more treatment image sensors generating treatment image data (serial static images or video). The recovery room also has one or more recovery image sensors generating recovery image data.

The computer processor and data store (memory and database) is coupled to the audio and image sensors to receive presence sensory data, treatment image data and recovery image data. When patients transition from a treatment room to the recovery room, the system and computer-based method generate patient unique treatment timing data based upon the presence sensory data. The system captures the treatment image and recovery image data as patient unique treatment/recovery image data and patient unique treatment/recovery timing data when the patient exits these rooms. The system digitally tags and segments the patient unique treatment/recovery image data based upon the timing data to generate time-stamped patient unique treatment/recovery image data, respectively for each type of room. This data is a series of static images or a predetermined time-limited video clip. Upon a replay time command from the designated patient or healthcare provider, the system and method displays the images or video clip.

An enhanced system (a) detects voice or visual presence of treatment patients, recovery patients and waiting patients at the HC-FAC and (b) captures image data of the treatment patients and recovery (serial static images or video). The system detects the presence of waiting patients with a sensory subsystem and monitors patient-unique wait times by detecting a first change in patient status when a waiting patient transitions from the waiting room to a treatment room based upon the sensory subsystem detecting both the presence and absence (exit) of the patient in the treatment room. The system generates treatment patient timing data and captures patient unique treatment image data for that patient. A second patient status change occurs when the patient transitions from the treatment room to the recovery room based upon the sensory subsystem detecting patient presence. This generates recovery patient timing data and patient unique recovery image data.

The stored data store is used to digitally tag and segment the patient unique treatment image data (the raw data) based upon patient unique treatment timing data to generate time-stamped treatment image data. The same process is used for recovery data to digitally tag and segment patient unique recovery image data based upon the patient unique recovery timing data to generate time-stamped recovery image data. The time-stamped treatment/recovery image data constitutes a series of static treatment images and predetermined time-limited treatment video clips (clips with predetermined run time, which run time can be reset by operator controls). Upon a replay command from the patient or healthcare provider, the system permits transmission to computer devices to display time-stamped treatment/recovery image data.

The basic multimedia medical documentation process and presentation platform uses the treatment/recovery image data (serial static treatment/recovery images and treatment/recovery video clips). These serial static images and video clips all have time-stamped patient unique treatment/recovery data associated with the digital media. The presentation platform is a display monitor with a user interactive display controller. The multimedia medical documentation process substantially simultaneously displays partial views of visual representations of medical data consisting of (i) serial static patient unique treatment/recovery images, and (ii) patient unique treatment/recovery video clips, and, a substantially simultaneous display of full or partial views of respective digital tags for the serial static patient unique treatment/recovery images and patient unique treatment/recovery video clips. The visual display of digital tags on the presentation monitor permits the operator to select one or another of the multimedia images or clips.

Also, as another enhancement, the system may include a process flow user interface (UI) to track patients as they move through the HC-FAC. With the wait/treatment/recovery room presence data, the UI is a 3 column, user interface display visually segmented into at least three columns (in a preferred embodiment, a five (5) column display is used) having (a) an arrived column displaying patient “tiles” who have arrived at the facility, (b) a treatment and recovery room column displaying patients in respective treatment rooms and the recovery room, and (c) a check-out column for the HC facility. Each patients is represented on the UI by a respective patient display tile and, as the patient moves through the HC-FAC, the tile is moved, either under operator control (for example, touch screen monitor control) or automatically by the AI system, to display the patient as he or she moves in the HC-FAC.

### BRIEF DESCRIPTION OF THE DRAWINGS

FIG. 1 diagrammatically illustrates a patient-centric process, capturing significant digital data, for a patient at an HC-FAC.

FIG. 2 generally illustrates one type of data acquisition or methodology to handle the generally raw data that is obtained during the data acquisition.

FIG. 3 shows various inputs, including audiovisual AV data, voice V data, voice commands v-cmd, and real time tags tag-rt, which are chronologically related.

FIG. 4 shows automated and manual curation of image (Img) and audiovisual (AV) data.

FIGS. 5A, 5B and 5C diagrammatically illustrate compilation of: a patient diary or data album, a medical education AV and image album for HC providers, and creation of medical materials for general population (Gen-Pop), respectively.

FIG. 6 shows post-processing of the various AI Img, AV, and e-text data collection from AI output 68 in FIG. 3.

FIG. 7 generally shows data flow for the system.

FIG. 8A diagrammatically illustrates the patient workflow scheduling and room manager display (a user interface UI or dashboard (panel)) and FIGS. 8B, C, D, E and F diagrammatically illustrate an automated notification system and process for each vertical panel in FIG. 8A, representing a communication action at substantially the same time, “Comm at T1.”

FIG. 9 shows the multimedia documentation prestation or storyboard as another aspect of the present invention.

FIG. 10 shows key performance indicator (KPI) process and methodology for the HC operation.

FIG. 11 diagrammatically shows typical hardware discussed earlier in FIG. 1.

### DETAILED DESCRIPTION OF THE PREFERRED EMBODIMENTS

The present invention relates to a patient-centric eco-system with an automated workflow (a Process Flow) and facility manager for improved delivery of medical services using artificial intelligence AI to curate volumes of acquired digital data, generating key performance indicators to improve efficiency and lower costs, and a multimedia medical documentation process and presentation platform for the compact display of the captured and curated healthcare data. Abbreviations used herein are listed in the Abbreviations Table near the end of this document.

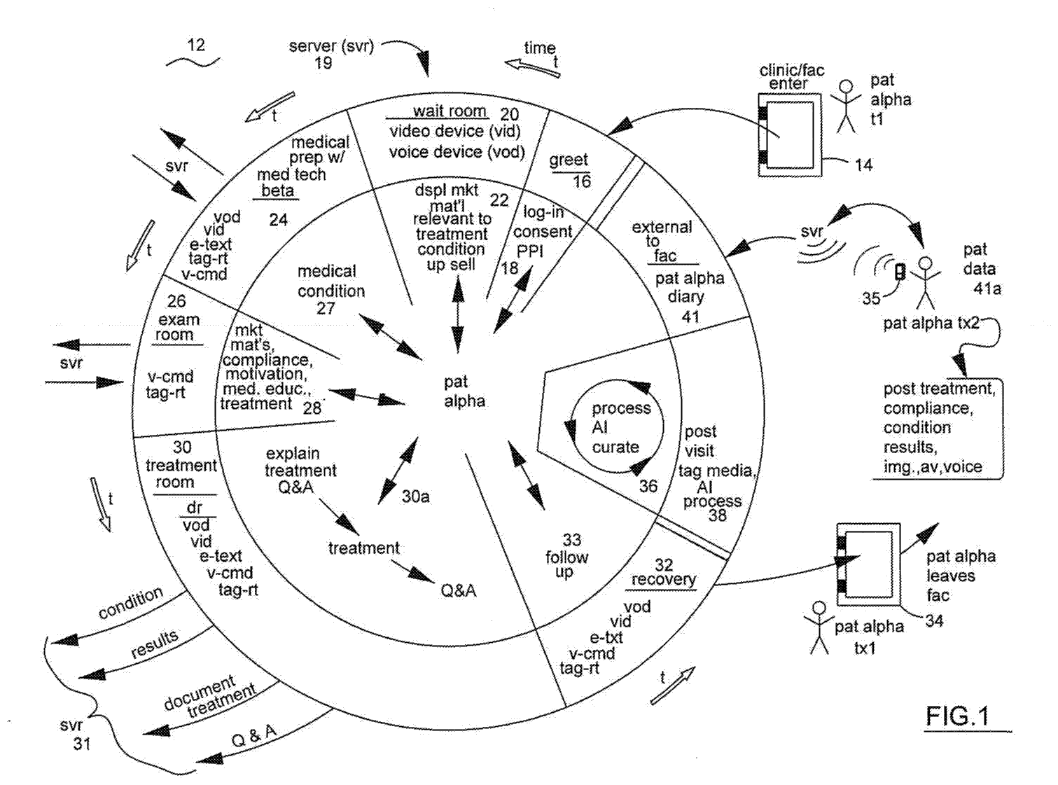

FIG. 1 diagrammatically illustrates a patient-centric process 12 for patient Pat Alpha. Pat Alpha at the time t1 enters the clinic or facility door 14 and is greeted at functional event block 16. At the reception desk, the patient is logged into the FAC as well as logged into the patient data records at function (fnc) 18. If needed, patient consent is obtained at fnc 18 as well as personal profile identifying PPI information. The HC-FAC is sensory rich with audio and video sensors (including static image cameras or sensors and video cameras or sensors). As time t progresses counterclockwise in FIG. 1, Pat Alpha occupies the waiting room 20. The VOD/VID detects the presence of waiting patients as part of the sensory subsystem and the processor and data store or memory monitors a patient-unique wait time for each patient. The patient-unique wait time is stored with other data by the processor. The waiting room has a voice activated device VOD (a voice monitoring and response device) or a video interactive information device VID (a display with a speaker and microphone). Further, the waiting area has display monitors 22 which may show marketing materials to patient Alpha. Also, patient Alpha may be presented with information via the display monitor 22 relevant to his or her treatment and his or her current condition and may be presented with information for additional services or products (an upsell event). The VOD may be similar to an ALEXA™ system. These VOD units and VID units form the sensory rich HC-FAC. These VOD units and VID units form the sensory subsystem of the computer-based methods described herein. For each treatment room, recovery room and waiting room, the sensory subsystem (a) detects voice or visual presence of patients to be treated (treating room presence data), patients in recovery (recovery room presence data) and patients waiting in the waiting room (wait room presence data), and (b) captures image data of treatment patients and recovery patients (patient unique treatment image data and patient unique recovery image data). This “image data” consists of a series of static images or a video stream. The image data from the VID (which VID may be a video camera and/or one or more static image photo cameras) may be video (visual data plus an audio track) or static images.

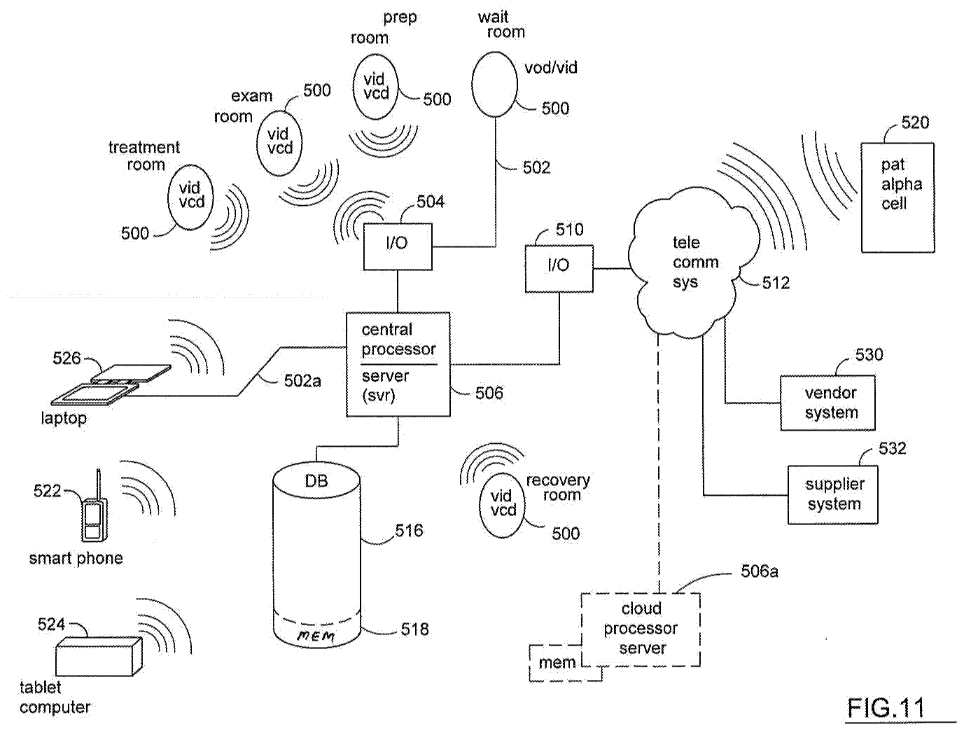

After the waiting room, patient Alpha is directed to the medical prep room 24 by a medical technician Beta, an HC staff member. Another VOD or VID is active in the medical preparation room or area such that the medical technician can enter electronic textual E-TXT material via his or her tablet or laptop and/or provide voice commands “v-cmd” which are sensed, recorded and acted upon by the server (SVR 19) or central computer at the facility to ascertain the presence of Pat Alpha in the exam prep room. See FIG. 11 system diagram. This transition of Pat Alpha from the waiting room to the medical prep room is a change in patient status. The sensory subsystem electronically notes that the patient left the wait room and entered the medical prep room. There is a patient unique prep room timing data (and a wait room exit timing data) noted by the system. Medical condition data 27 is obtained. As discussed below, the system may be deployed as a cloud-based system rather than a central computer server at the facility. Typically, the HC-FAC would have both a local server and a cloud-based server and data store. Further, the VID may record Pat Alpha in the medical prep area.

Subsequently, patient Alpha is taken to the sensor rich exam room 26 which again is provided with VOD or VID equipment. This change in patient status is an auto-capture event by the VOD/VID. This change in patient status is noted by the system with prep room exit timing data, exam room entry timing data. The system then calculates the patient prep room time-based data. The HC staffer may enter an additional E-TXT information via a laptop (see FIG. 11) or other type of computer entry device in the exam room as. The HC staffer may take a photo image of a particular body part to be treated and upload the Img image to the server. The photo has a time stamp that is used by the system to integrate that photo into a composite diary with other images captured in the exam room by the VOD/VID. The HC may further provide voice commands v-cmd to the system. For example, “System Photo now” v-cmd while the staff takes an image. The system automatically recognizes when patient Alpha or the HC has entered the room (based upon audio detection or video movement detection, a presence event) and provides a real-time tag “tag-rt” to the data stream generated by the VOD-VID. The tag-rt can be used to time-base or synchronize and match images captured by different devices in the exam room. The voice generated v-cmd “Photo now” creates a digital data tag command that can be used to time-mark the time of photo Img when both the Img and the VOD-VID data is merged together in a composite by the AI system. In the examination room 26, patient Alpha may be presented, via a display monitor, marketing materials 28, information regarding post treatment compliance 28 (post-treatment use of drugs, lotions, exercise) and other helpful information 28. Patient Alpha may also be presented with medical educational materials and information 28 about his or her treatment.

After the examination room, patient Alpha is taken to the treatment room 30. Another HC, possibly a doctor, provides treatment to patient Alpha at that time. The treatment room also includes VOD-VID equipment (for the presence data and the patient unique timing data (entry and exit)) and, most likely, other computer devices permitting the doctor and other HC staffers to input data (E-TXT) into compiled records based upon real time rt markers in the digital data Img and the date/time stamp on the video. Still images Img may be taken of the treatment site with a concurrent v-cmd command to one or more VID devices or, alternatively as a time-based marked to the system t1 note capture of Img by another digital device. The VID may capture a video clip (e.g. 10 sec) time-marked with a v-cmd (the system pre-set to capture a 10 sec. clip upon v-cmd VIDEO ON, the clip run time can be changed by an operator control setting). The doctor may provide voice commands and the VOD-VID will tag data streams with a real time tag-rt based upon the voice commands v-cmd or based upon other events. For example, if a special treatment light is activated to highlight a surgical field, the VOD-VID may react to this special light (a wavelength sensor) and the server or central computer or cloud-based system tags that data stream with a real-time tag tag-rt based upon sensing of that special surgical light and the time-data stamp on the video data block. Electronic ON-OFF data links from medical equipment may also be acquired data by server 19. This is similar to operation of IOT (internet of things) enabled devices. As noted in block 30a, the HC provider can document his or her treatment explanation, record treatment data and respond to Pat Alpha in a Q&A.

The data flow from the treatment room includes the patient condition, the results of the treatment, electronic data to document the various events that occurred during treatment, and records of questions and answers exchanged between the patient, the HC and/or the doctor. See server interaction 31.

After leaving the treatment room, Pat Alpha is placed in the recovery room 32. This is another change in patient status when Pat Alpha transitions to the recovery room. Patient unique recovery timing data is collected at each transition in the HC-FAC. The recovery room also has a VOD-VID and potentially other ESI (electronically stored information) devices such that HC can enter electronic text and information (E-TXT). The VOD-VID captures voice commands and provides sensory inputs for real time tagging of the data flow. Follow-up 33 is provided in room 32.

Although several rooms are discussed in connection with FIG. 1, some of these rooms may be combined together. Also, additional rooms or HC spaces may be found in the HC-FAC. The VOD-VID can be used to recognize the HC or doctor when he or she enters the room if the HC or the doctor announces himself or herself (voice recognition based upon the provider's name and the patient's name, pre-programmed into the system earlier). The computer system coupled to the sensory VOD-VID devices, recognizes the name of the HC of the doctor. Facial recognition by VID sensory devices may also identify HC providers in these rooms (the system having earlier captured the provider's image and the patient's facial image to match the room-to-room transition and change of patient status). As a result of these data acquisitions (entry and exit time-notes events), a real-time tag is added to the acquired data stream. In one embodiment, the VID is the master data acquisition device, keeping a time-based track on all events in the designated room. A V-cmd may be used to annotate and collate other data streams from digital collection devices, such as cameras taking photo Imgs, laptops used to input E-TXT by scribes or the HC providers, equipment sensors, medical devices creating digital data, light-activated devices, etc. Otherwise, the HC or the doctor could issue voice commands v-cmd which mark the events in a designated room with Pat Alpha or turn ON or OFF video cameras, or still cameras. These are digital tags used to segment and curate data streams from each room in the HC-FAC.

Following the recovery room, Pat Alpha leaves the facility (door 34) at tx1 and thereafter communicates with the computer system servicing the HC facility via the patient's smart phone or cellular phone or computer tablet 35 at tx2. In this posttreatment, at time tx2, the system is focused on posttreatment conditions of the patient, compliance with drug, exercise or food intake, the post-op condition of the patient, the results of the treatment, and other medically related information. Prior to leaving, Pat Alpha would check-out of the HC-FAC. While away from the HC-FAC, Pat Alpha communicates with server 19 via a common telecomm system and receives post-treatment advice and motivation in block 40.

Shortly after Pat Alpha leaves the facility, the system processes the data captured for the patient and utilizes artificial intelligence and enables the system operator or system users to curate the data in fnc block 36. All this is done after the patient visits the facility. Generally, media and data streams are tagged and segmented in fnc block 38 and further processed in a artificial intelligence AI curation process. ESI med data is tagged, chronologically stored and processed for Pat Alpha under his or her PPI. The result, as described later, provides an ESI diary 41 for Pat Alpha not only for his or her visit to the facility but also for posttreatment events and post-op treatment plans (block 40). Pat Alpha can add data to the ESI diary by acquiring digital data on here or his own time and uploading it to computer system svr 19 servicing the facility via the patient's cellular telephone 35. Later on, Pat Alpha can download the ESI diary as needed.

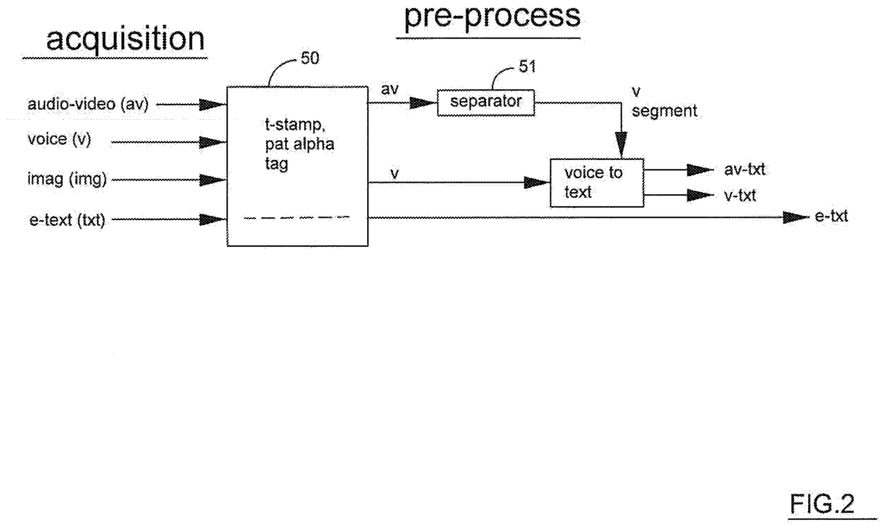

FIG. 2 generally illustrates one type of data acquisition or methodology to handle the generally raw data that is obtained during the data acquisition. When patient Alpha is at the facility, audiovisual information (AV) is one type of input, another type of input is voice (V) data, a third type of input is image (Img) data, and a fourth input is electronically input data typed in by the HC or the doctor as (E-TXT) data. This raw data is chrono or time stamped by the system and further the raw data is initially processed with patient unique tag, fnc block 50. This data is preprocessed such that the audiovisual data is sent to a separator 51 which generates just the audio portion. The voice segment v-segment from the AV data is applied to a voice to text converter (av-txt). The voice or audio raw data is also applied to the voice to text converter (v-text). The output of this conversion is the ESI text for the audiovisual AV-TXT and the electronic ESI text for the voice raw data V-TXT. Of course, the electronically entered data E-TXT from the provider's laptop or tablet is also available.

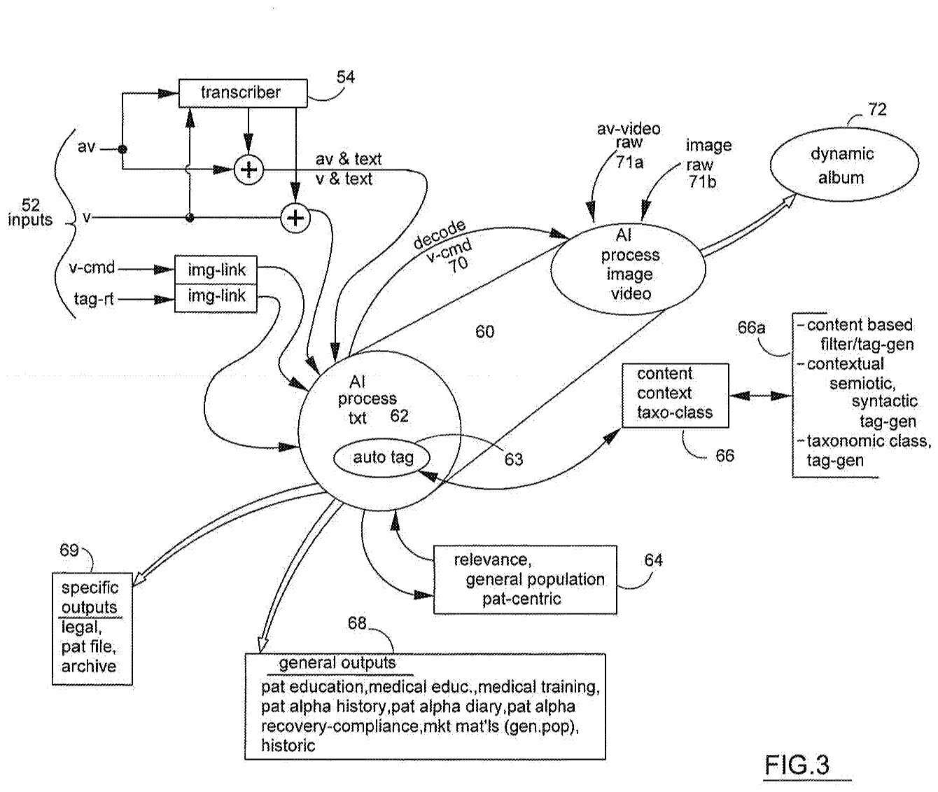

FIG. 3 shows inputs 52 which include audiovisual AV data, voice V data, voice commands v-cmd, and real time tags tag-rt which are chronologically related. The audiovisual material is applied to a transcriber 54. The raw voice V material is also provided to the transcriber. The electronic text representing the voice track on the AV material and the electronic text for the raw voice V material is added to the data stream. The resultant AV plus AV-TXT and V plus V-TXT and the E-TXT data is fed to an artificial intelligence AI processor 60.

The voice commands v-cmd are chronologic markers to be used to link data streams or Imgs from other digital acquisition devices at the HC-FAC, hence these chrono markers on the VID/VID data stream are at the nearly identical time on the AV data stream and/or the V datastream as the data streams or Imgs from other digital acquisition devices. The same thing is true regarding the real time tags tag-rt which may be added to the VID/VOD data stream as time-based markers by a provider v-cmd.

The v-cmd and tag-rt are also supplied to the AI process for textual processing 62. The text is supplied to a relevancy processor 64 to identify to whom the resulting post processed information may be significant. Sometimes, information is only significant to a particular patient such as Pat Alpha. This ESI is linked via the database to Pat Alpha PPI. At other times, the information is relevant to the general population (Gen-Pop) whose members may undergo a similar treatment. Further, the information may be used for a group of similarly situated patients similar to Pat Alpha (a patient sub-group). Further operational details of the relevancy processing 64 are described below.

The AI process also activates an engine 66 which analyzes the content of the electronic text (txt, a combination of AV-TXT, V-TXT, v-cmd, tag-rt, and E-TXT), the context of the words and phrases in that combined electronic text, and provides taxonomic classification of certain words in the combined electronic text. This taxo-class results in an electronic tag or label for the electronic data. With respect to content processing, the engine may utilize a content-based filter and generate tags unique to particular words or phrases explicitly located within the electronic text. A table of special words may be used to match text in the combined ESI. The engine 62 may also look at contextual relationships between words, semiotic relationships, syntactic relationships and generate data tags 63 unique to the textual, semiotic or syntactic relationships. See fnc 66a. The engine 62 further identifies hierarchical superclasses and the hierarchical subclasses for the combined ESI electronic text as processed by the content filter and the contextual filters.

The AI process automatically tags fnc (block 63) these ESI data streams, both the electronic text and the AV data stream and the audio V data stream and the image Img data and Img sequences (a series of Imgs taken over a short period of time, e.g. 10 sec.). Long data streams, such a constant continual RECORD ON by a VID device, are segmented or separated base don the time-based markers. For example, a v-cmd RECORD SURGERY ON may capture 30 minutes of time, but tag-rts and other v-cmds in this long data stream can be used to segment, for example, 10 sec. video clips from the long data stream. The resulting data can be separated or segmented automatically by the AI engine, content, relevancy, context and taxo-classification tags, and further curated by the user or Sys Oper into General Outputs 68 for patient education, medical education, medical training, Pat Alpha history, Pat Alpha diary (the patient diary is made available to the patient as requested by the patient at fnc block 41), Pat Alpha recovery and compliance 40, marketing materials for the general population 22, and for historic purposes unique to the doctor, the facility and the HC staff. Further, the AI process generates specific outputs 69 for legal purposes, patient record files and as an archive.

Returning to the AI process 60, the AI decodes the voice commands v-cmd (fnc block 70) and accepts the audiovisual raw data and the image raw data (inputs 71a, 71b). The AI processes then integrates, the AV data segments based on the voice commands and further indexes the AV data based upon the content, contextual, taxonomic classes in the relevancy process 64 for the general population, for patient-centric purposes or other purposes. The same type of processing occurs with the image data. The result is a dynamic album 72.

In FIG. 3, the AI process block 60 may analyze content, context and taxo-classification in fnc 66 (with filters (context), matching word/phrases (contextual), and hierarchical taxonomic classifications (sub-classes and super-classes)) before or concurrent with the relevance fnc 64. Also the HC provider may issue a v-cmd to designate in real time a relevancy factor (like a point of interest, POI marker) that is recognizable by the AI process 60. For example, the HC staff/provider may use a v-cmd, such as “interesting POI Gen Pop” as a key point of interest which the AI v-cmd fnc 70 decodes as “mark image/10 sec video clip segment” as “Gen-Pop relevant.” Of course, the POI v-cmd may designate an in-house med or medical educational POI, a general med educational POI, a marketing POI, a Pat Alpha POI for the PPI, a Pat Alpha Diary POI, a note-to-file POI, etc.

As for content detection fnc 66, the medical field consistently documents common words and phrases and the AI fnc 60 and fnc 66 accesses these medical term dictionaries. The AI system would be trained using this data set from medical term dictionaries or medical documents. The context fnc 66 is the relationship between words/phrases in a part of a sentence compared to other words/phrases in another part of the sentence which, when juxtaposed, give meaning to the concept in the larger phrase or sentence. As for the taxo-class fnc 66, the medical profession has dictionaries and language patterns permitting hierarchical taxonomic classification identification to higher level super-classes and lower level sub-classes which are related to the event or condition evidenced in med rooms 24, 26, 30, 32. As for the relevancy fnc 64, physician specialists understand the words and phrases used in their respective speciality, hence a relevancy category for in-house med education and physician med education can use these special med terms and the terms can be assigned by AI 60 to the series of data blocks captured in, for example, treatment room 30. The AI 60 is trained to detect lower level sub-classes used by physician specialists and auto tag data blocks in fnc 63. However, for marketing materials to be used in waiting room 20 or recovery room 32, higher level super-class words and phrases can be assigned as a “relevancy category” by the AI fnc 60, via auto tag fnc 63, to the acquired data block. The foregoing represents examples of pre-processing of training data (e.g., preparing unique datasets for input) into the AI algorithm. Other training sets can be gathered by others in the HC field. Other AI algorithms may also be used to identify ESI data, AV data and audio data acquired by the AI system 60.

As for training AI fnc 60 (see FIG. 11, processor 506, database 516, memory 518, cloud-based processor and memory 506a), the initialization of the AI system may have the HC staff/provider audibly recite words linked to preprogrammed v-cmds concurrent with the display of the associated computer command or the HC staff/provider may download medical dictionaries into the AI system (as a gross example, the Merck Medical Dictionary), the staff/provider then selects the sub-category of his or her speciality, and the AI system displays the medical term for the v-cmd and the staff/provider then audibly recites the word and the system records the word as the v-cmd.

On the fly training of the AI system when the staff/provider is in a med room is also possible, such the staff/provider audibly announcing an initial v-cmd “COMMAND [operator states proposed audible v-cmd, for example ‘wound’ ]”, then the AI system repeats the recorded v-cmd as V-COMMAND WOUND, and then the staff/provider confirms V-COMMAND WOUND RECORD. Thereafter, in all med rooms, the AI system captures, for example, image data acquired in a 10 sec time block and/or a 10 sec video data block by recognition of COMMAND WOUND. The capture time (10 sec) is variable by the Sys Op.

On the fly AI training for v-cmd can take place with a medical scribe assisting the staff/provider. The staff/provider would say V-COMMAND and the scribe creating the E-TXT notes the v-cmd with the next word or phrase announced by the staff/provider.

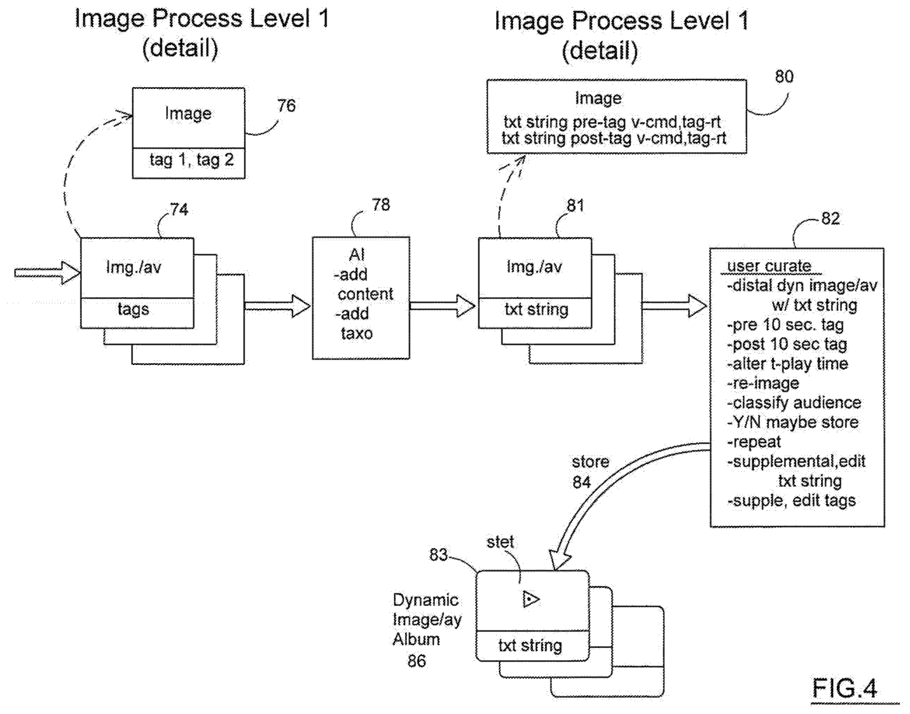

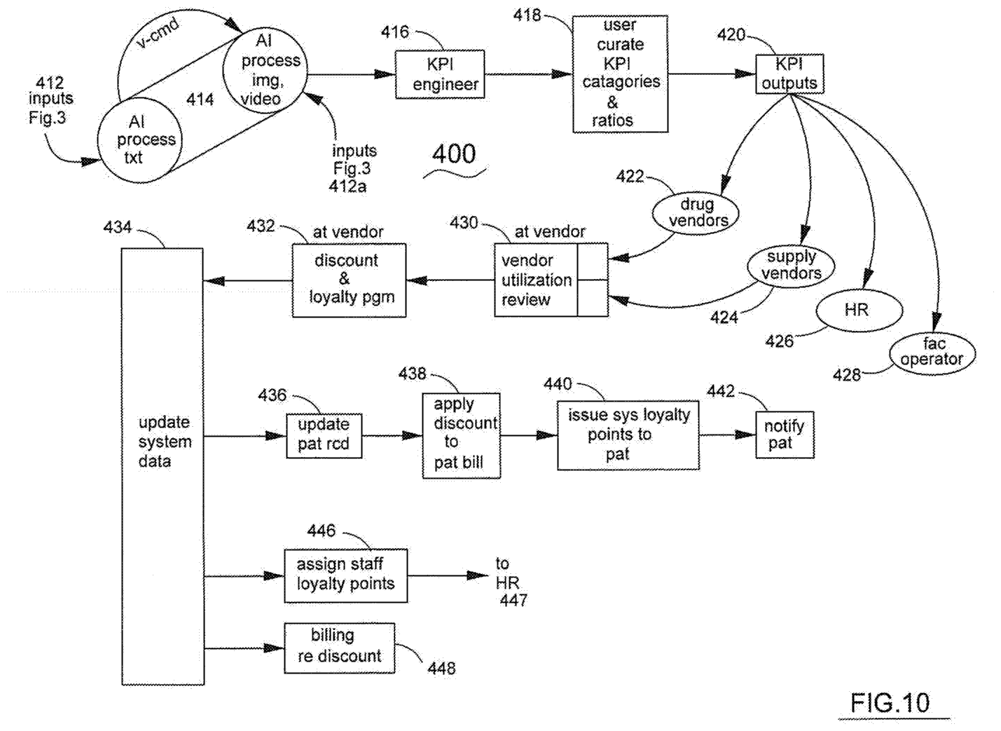

The AI system can be trained during usage in a similar fashion for content, context and taxo-classification (fnc 66) and relevance tags (fnc 64). The training process typically includes curation fnc 82 discussed in connection with FIG. 4. Other training processes involving real time improvements or adjustments to the AI engine use information and techniques known to persons skilled in the HC industry. To normalize the professional or worker classifications for patients in order to train the KPI AI system in FIG. 10, an initial training data set may use the Bureau of Labor Statistics (BLS) occupational classification system to classify patients into different categories. Hence, the examples herein only discuss some basic training processes.

It should be noted that the curation fnc 82 (FIG. 4) and 418 (FIG. 10) may use discriminator training which is supplied with randomly obtained real (genuine) outcome training results and fake (improper) outcome training results. As an HC example for a reasonably well healed wound image, the real input training result (used as an input to the discriminator network) includes images of many well healed wounds (taken at different angles, under different lighting conditions, etc.). The fake/improper input training result are not well healed wound images. If the discriminator network marks an output as “fake/improper”, that image is further processed through another network using noise plus the fake/improper image as a feedback. The output of the noise+fake/improper image network is then supplied back to the discriminator network until the discriminator output of improper wound images is below a preset threshold. This AI training process is sometimes called a generative adversarial network (GAN). The GAN model can be used to generate or output new examples that plausibly could have been drawn from the original dataset. The GAN is a supervised learning process with two sub-models: the generator model that trained to generate new examples, and a discriminator model that tries to classify examples as either real (from the preferred result of collection of preferred results as a data domain) or fake (generated and not preferred). The two models are trained together in a zero-sum game, adversarial manner, until the discriminator model is fooled about half the time (a 50% threshold), meaning the generator model is generating plausible examples.

These AI processes may also use a disentanglement network. The AI system can learn to pick apart a scene into the objects that constitute it. One network compresses the input data of common shapes and the unknown wound image (the common shapes being well healed wounds) and the other network expands them again. By constricting the link between the two, the AI system is forced to find the most parsimonious or best-fit description or character classification of the wound under study.

Since the process flow system and method employs VID in med rooms 20, 24, 26, 30, and 32, the AI system can use facial recognition to physically track patients and HC staff/providers enterng and exiting each med room. One method to train the AI system 60 (FIG. 3) to recognize faces is to take one or more photos (images) of all the HC staff and providers and, upon the initial intake of a patient, to take a photo (image) of the patient. This staff/provider image data is logged into the HC HR (human resources) data collection and the patient data is also recorded with the PPI for the patient. Prior art facial recognition software and methods can be used by the AI system 60 to physically track HC workers and patients as they move about the HC-FAC.

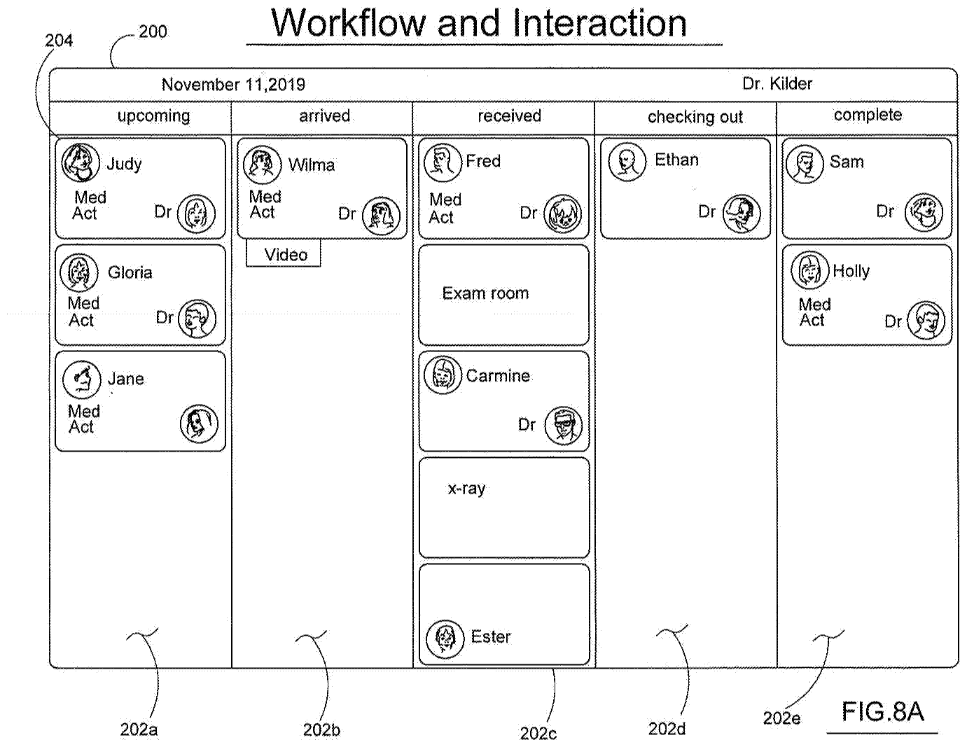

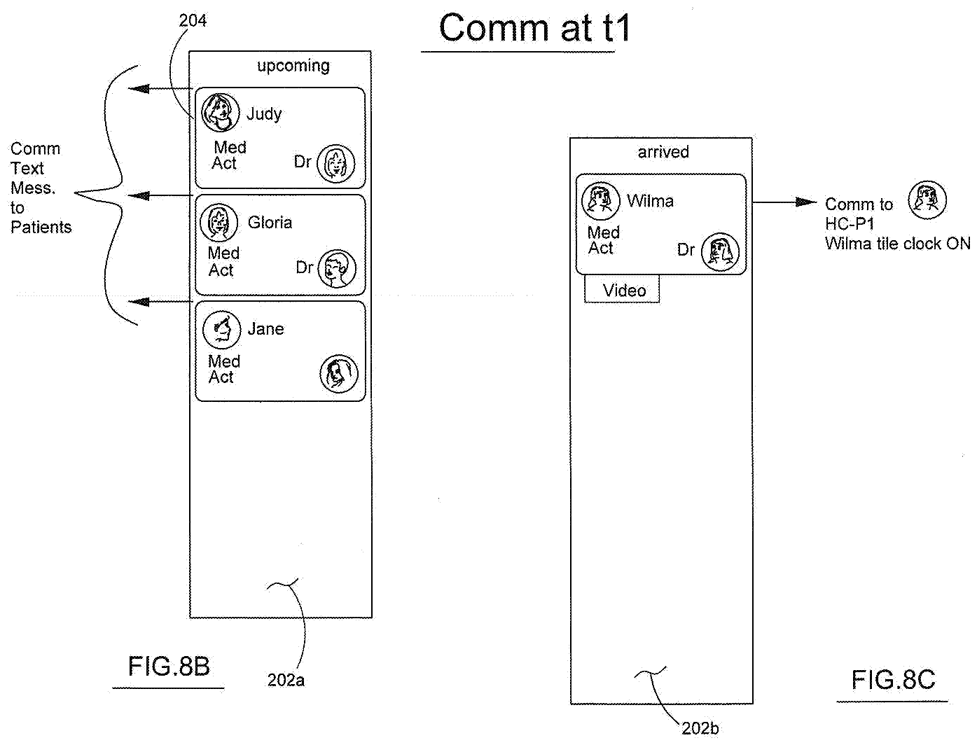

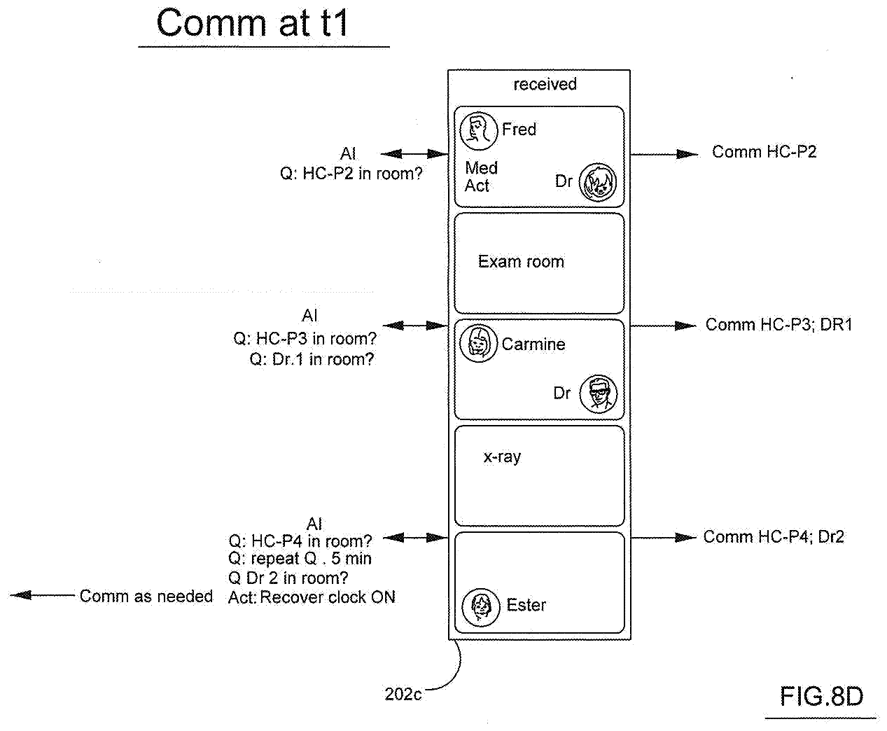

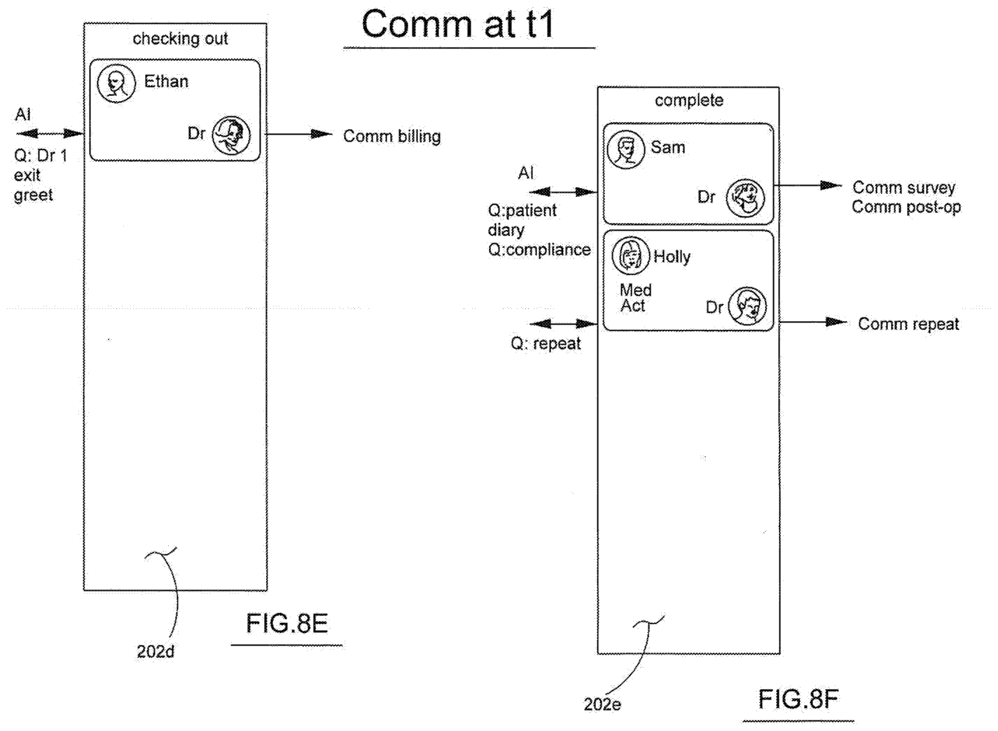

As noted below, when a patient moves or transitions from one HC space or room, the authorized user of the process flow user interface (UI) in FIG. 8A “moves” the patient tile on the UI to another HC space or room. The AI system confirms the operator's UI display input (e.g., a touch screen monitor), records that UI patient tile action and confirms the patient's change in status from room A to room B in the HC-FAC. The AI system may include a feedback loop to confirm the Pat Alpha was physically in the UI designated room or space, either before the UI patient tile action or after the tile action. The AI confirm may be time delayed to account for the physical movement of Pat Alpha. If the AI sensory system does not note that Pat Alpha is physically in the UI designated space or room within a pre-set time frame (a countdown from the UI operator tile movement), the AI system (a) visually alters the patient tile (e.g., flashing, highlighting, etc.), (b) communicates with another authorized user of the UI process flow; and/or (c) communicates with the HC staffer most recently interacting with Pat Alpha (e.g., the last provider in the last HC room withe Pat Alpha). The AI system escalates these “UI/Patient Movement Error” communicative notifications to HC management as the time differential between the UI tile action and the “confirm location” of Pat Alpha increases. In a highly trusted AI sensory rich system, the AI may move the patient tile only when the designated patient is in the next HC room or space associated with the patient's treatment regime. These comm notifications are generally identified as independent communication notifications to one or more of healthcare providers by the processor, data store and telecomm network based upon patient unique room presence sensory data. For example, waiting room presence sensory data exceeds pre-set-wait-per-patient period, prep room presence sensory data time period exceeds pre-set patient-time-to-provider-arrival period, exam room time period thresholds, treatment room thresholds, and recovery room thresholds, all patient unique treatment room presence sensory data, and patient unique recovery room presence sensory data.

FIG. 4 shows that the image Img and or the audiovisual av data now has a number of digital tags (like meta tags) relationally associated therewith in a typical database configuration. See block 74. Further, the image and/or the audiovisual data is segmented under automation by the AI processor 60. There are several ways to configure the database, for example, the entire VID data stream may be saved in one data storage area and a time index database may be used to locate, copy and extract video segments from the complete data stream. Otherwise, video clips may be stored in another data store for fast retrieval or due to HIPPA security protocols. A detail of image 76 is shown as an excerpt from the main data stream by dashed lines to Img 76 in FIG. 4 such that the image 74 has several tags, tag 1, tag 2. These tags may indicate a pre-op condition (tag 1, pre-op tag-rt) and reference the use of image data 76 as for general population or other relevance category. When the data is marked for general population, marketing or med education, all the personal identifying information PII is stripped from the electronic data or blocked from the image. For example, only a detailed image of a wound site may be marked Gen-Pop, etc. (the detail PII data imagery extracted or redacted from a larger image of the patient).

After the tagging, the AI processor adds contextual tags and additional taxonomic tags at 78. The resulting image or audiovisual material now has a text string TXT-Str that is associated with the data block image, video clip or shortened datastream and these digital tags are converted into visual representations and are visibly presented in the Img or video clip similar to a photo caption. For example, the image processed to a detail level 2 (block 80) shows that the image has a text string TXT-Str for a pre-op tag voice command, a first real time tag, and a post-op voice command tag and a second real time tag. Tag-rt-1 is earlier in time than tag-rt-2. The v-cmd pre and post-op commands then point to video or images at tag-rt-1, tag-rt-2. These are time-stamped treatment image data and time-stamped recovery image data. Other pre-op, exam and diagnostic digital data is similarly processed as time-stamped data. Serial Img images are a series of static treatment images, for example, 10 static images of a wound during surgery.

After the image or AV material contains a text string TXT-Str (the text string being, for example, “pre-op Patient 20191104” or “post-op Patient 20191104”), the system permits a user to curate the segment 82 based upon txt-string tagged images at 81. This curation involves displaying the dynamic image or audiovisual material with the text strings to a HC user. The user may add an additional 10 seconds to the AV material (enlarging the data block) and add a tag as precursor tag to the datastream. Otherwise, the user may add an additional 10 seconds to the AV datastream along with a post-curser tag. The user may alter the playing time t-play and edit the length of the AV segment. The user can re-tag the segment as needed, thereby classifying an audience (Gen-Pop, in-house marketing video, med training). The user or Sys Op makes an initial determination whether to store the edited result, delete the edited material, or classify a “maybe use” or “return to” condition for that AV segment or image.

By repeating the editing and curation operation, a supplemental edited textual string TXT-Str is added to the image or AV material 83. The supplemental strings can be edited and additional tags added. Eventually, the data streams are stored 84 to provide a dynamic image and or audiovisual album 86. As indicated earlier in connection with FIG. 3, the user can curate for in-house patient education, medical education, medical training, patient specific history PPI, patient diary PPI, release 41, patient recovery and compliance PPI-release 40, marketing materials for the general population (Gen Pop) and may further curate these segments for historical reasons.

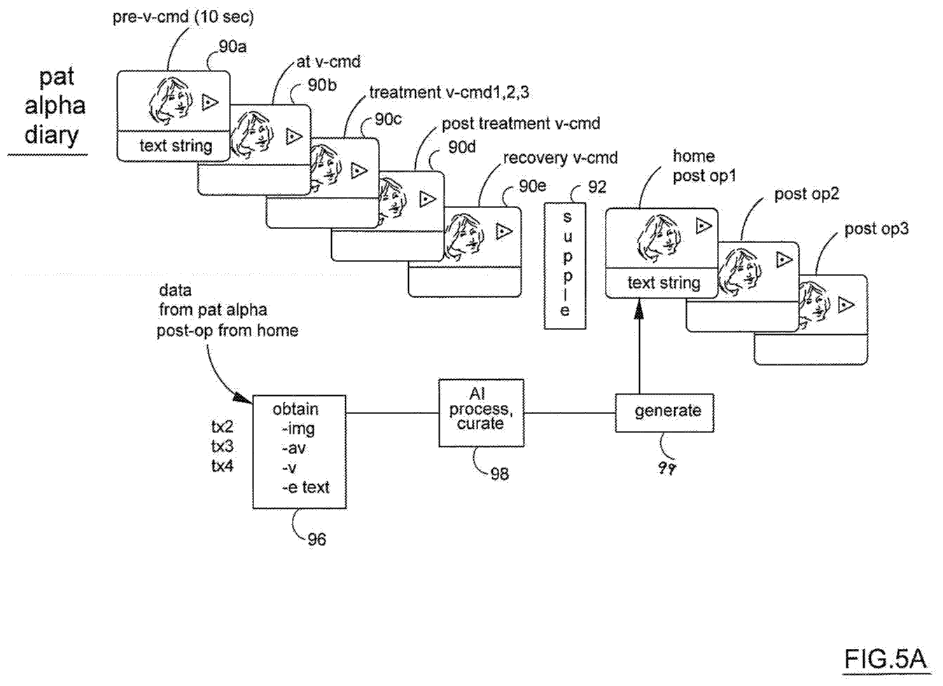

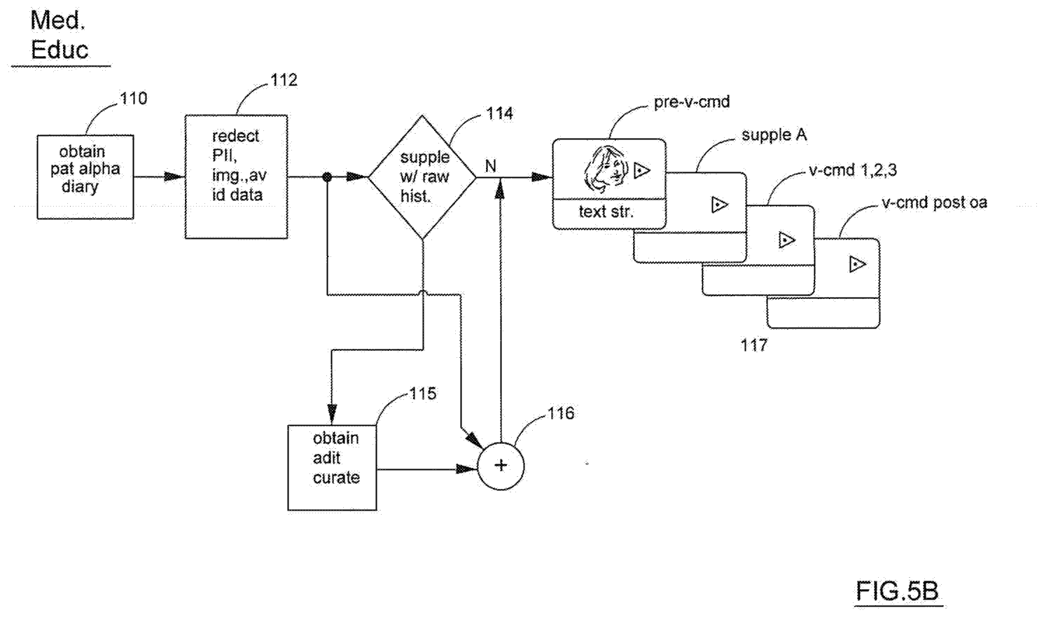

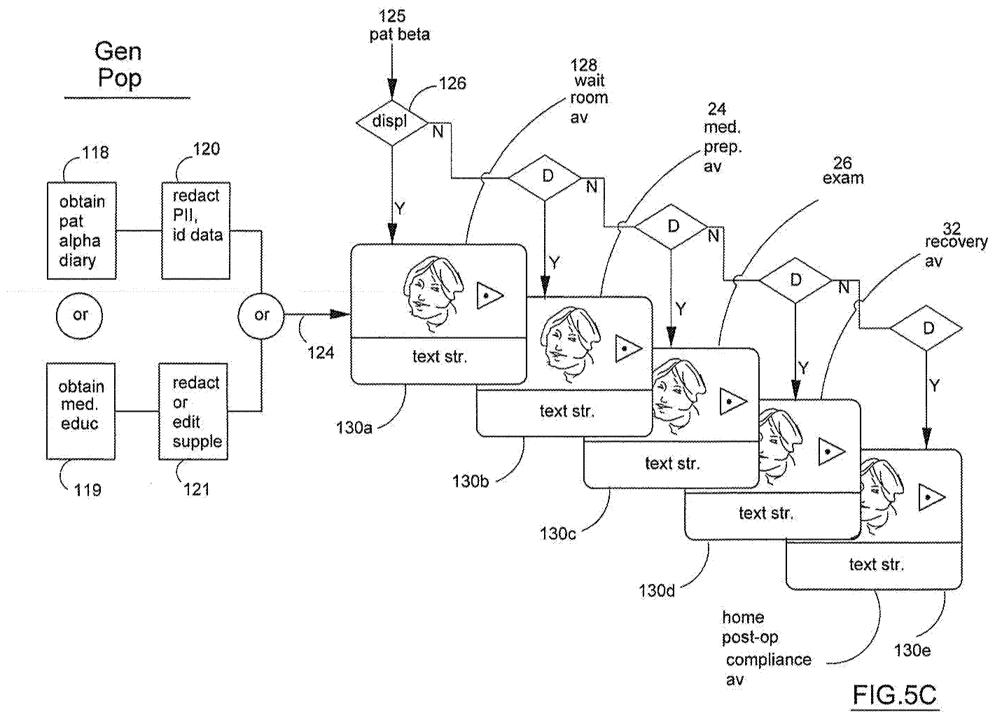

FIG. 5A diagrammatically illustrates a patient diary PPI-release 41 (FIG. 1) for patient Pat Alpha. The first block 90a is a pre-op data, captured at voice command, as a AV segment or material consisting of about 10 segments or 10 seconds with a text string that patient Alpha can see prior to playing the AV data segment (Pat Alpha sees the TXT-Str). The next AV segment 90b to the right is a three second AV data stream of block which can be played by Pat Alpha. The third segment 90c is a series of treatment videos which are AV segments triggered as “record ON” by voice commands 1, 2 and 3. The fourth AV datastream 90d shows posttreatment material captured by a voice command. The fifth available AV data stream 90e is the recovery AV segment captured by V-command Upon a replay command, Pat Alpha can visually review these portions of the patient diary composite.

Pat Alpha may want to supplement his or her patient diary material with data captured outside of the HC-FAC. Any patient specific data supplementation 92 is added to the patient diary 41. After Pat Alpha leaves the facility at door 34, he or she may post video or images into his or her diary 41 by uploading the images Img or AV material or textual material of voice V or ESI material into the FAC computer system. See block 41a, FIG. 1. Videos post-op 1, post-op 2 and post-op 3 are shown in FIG. 5A. Patient Alpha may upload these videos at block 94 into his or her diary and the system activates the supplement diary function. Images are obtained from the patient Alpha home at time TX-2, TX-3 and TX-4. For example, text message images 1, 2, 3 sent to the HC-FAC. The date on the text message is used by the AI system to time stamp the images. After obtaining the images 96, those images are processed via the AI process and curated 98 by the system operator. The textually marked material (marked with text-string or TXT-Str) is then generated 98 and placed in the patient diary 41 and is available to view as post-op 1, post-op 2 post-op 3. Pat Alpha can view the TXT-Str beneath each IMG or AV and therefore select which one of the several AV or Img are suitable for viewing on his or her cell phone, laptop or tablet computer. After insertion of the text strings or TXT-Str, these data streams are stored in the patient Alpha diary 41. Further, the supplements 1, 2, 3 for the patient Alpha diary are fed as inputs into FIG. 3 to supplement the base data for the Pat Alpha history and Pat Alpha compliance.

FIG. 5B shows a process for preparing medical education AV and image materials for other HC providers. The first function obtains patient Alpha diary 41 at block 110. Other Pat Alpha data may be selected as initial input data. The next function 112 redacts personal identifying information PII from the images and the audiovisual and any other identifying data. The following decision step 114 determines whether to supplement this partly edited AV/Img material with raw historic data (YES) or not (NO). If YES, the user obtains, edits and curates this historic data 115 and adds or supplements 116 the partly edited data to the materials in process. The result, either from block 114 or 116, for the medical education process, is an AV/Img data string with pre-op triggered voice commands including appropriate textual identifiers as TXT-Str, supplemental AV/Img materials (supple A) and AV/Img data blocks captured at voice commands 1, 2, 3 (v-cmd 1, 2,3) and a post-op voice command triggered AV/Img data block 117 (4 video or image data block, each with a visually presented text-tag TXT-Str). Of course, some of these AV items can be a single image Img rather than a time-based AV presentation.

FIG. 5C shows the creation of materials for general population (Gen-Pop). This process may begin with patient Alpha's diary 118 or may begin with the preprocessed medical education materials 119. If a patient diary 118 is utilized, the system redacts PII in any other identifying personal patient information 120. Some redaction is automatic, such as by “blacking out” the eyes and nose bridge of a patient and deleting any voice track revealing the name of the patient. Some redaction may be applied by user curation. If medical education data 119 is the initial data starting point, the system redacts or edits or supplements 121 that material to be more suitable to the general population. The resulting AV/Img collection at flow point 124 permits patient Pat Beta 125 to display (displ or D block 126) certain videos/images 130a in the waiting room 128 (block 20, FIG. 1). As described below, the authorized user of the Process Flow UI can play the waiting room video.

IfYES from block 126, patient Beta replays the data block and sees the AV material 130a as well as the TXT-Str. If NO, patient Beta does not view the material. Pat Beta in the medical preparation room 24 can select and display D the AV material 130b. The same is true in the exam room 26 (AV 130c) and also in the recovery room 32. Patient Beta also has a opportunity to see the post-op compliance AV material 130e in the recovery room 32 or at home 40.

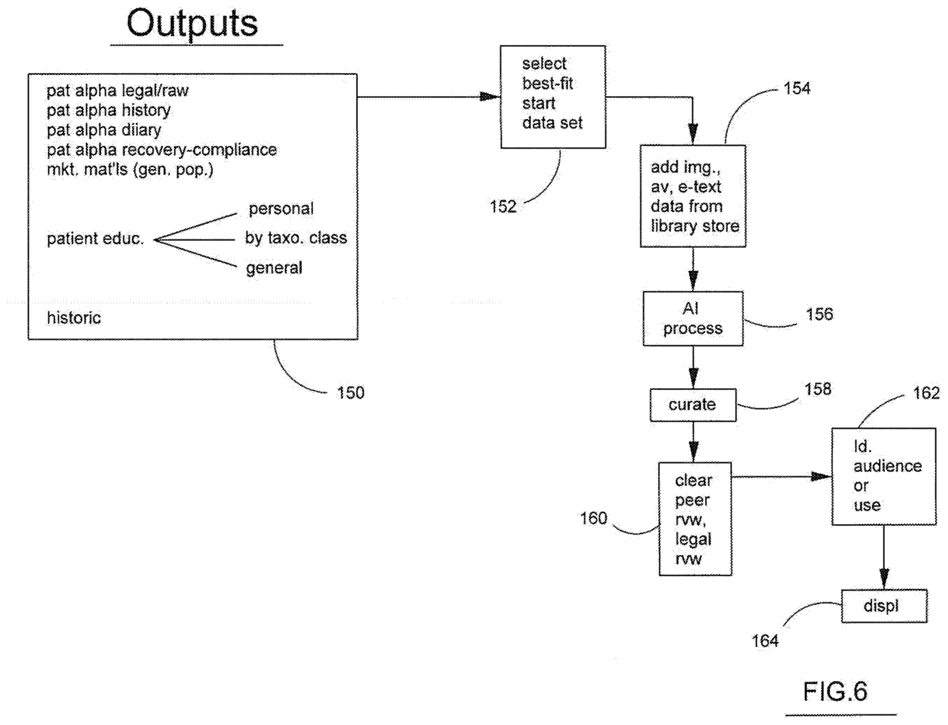

FIG. 6 shows various outputs which may be included in AI output 68 in FIG. 3. For example, these outputs 150 may include patient Alpha history, patient Alpha legal, patient Alpha raw material, patient Alpha diary, patient Alpha recovery, Pat Alpha compliance materials (drug and at-home post-op treatment), marketing materials for the general population, and patient educational materials (including personal materials unique to Pat Alpha) or materials for a taxonomic Gen Pop class, age group gender, race or category of employment. Also, historic data (archive) is stored without regard to a particular patient.

The user during the curation step and function 82 (FIG. 4) may select the best fit 152 based on time, target audience (identified in the previous paragraph), quality of content and audience feedback for particular AV data block or image data set. The user in the next function 154 can add images, and AV material, and electronic text data from the library store or type-in the material himself or herself onto the content. The next step 156 integrates an AI process to assist the user during the curation. For example, the AI process may identify a certain surgical event and suggest a more appropriate TXT-Str for the image or AV material. The next step 158 is curation by the user. The following step 160 involves clearance of the materials, that is the AV or image production data set, by peer review as well as legal review. The following step 162 identifies the audience or the use of the prepared material. The last step 164 involves display of the material to the selected audience. A s noted below, the AI system is integrated into the process flow method which gathers feedback from (a) post-op patients, (b) images of successful treatments, and can be configured to accept (c) audience feedback as detected by the VOD in the waiting room and the recovery room (the VOD monitoring eye contact on the display screens), and (d) peer review comments. These feedback loops are included in the curation fnc 82 as further feedback and corrective measures to improve patent experience.

Another benefit of having the AI data acquisition system, which is curated by the AI and by the Sys Op or HC professional, is that key point of interest POI materials can be quickly located in the digital storage system and shared to in-house professional or in-network professional to solve a problem encountered at locations remote from the HC-FAC. For example, if during a medical procedure, an in-network physician at a remote HC-FAC experiences a situation unique or generally not encountered for that procedure, that physician can seek help from his or her colleagues. By sending a text or critical email to the network, the Sys Op at the HC-FAC can quickly locate POI from similar medical treatments and view the procedure (by video captured data, curated by the AI and human interaction system) and share that POI data with the inquiring professional. In the absence of a dynamic data acquisition system, AI curation and Sys Op curation, one cannot quickly locate and share the corrective POI data block with the remote professional.

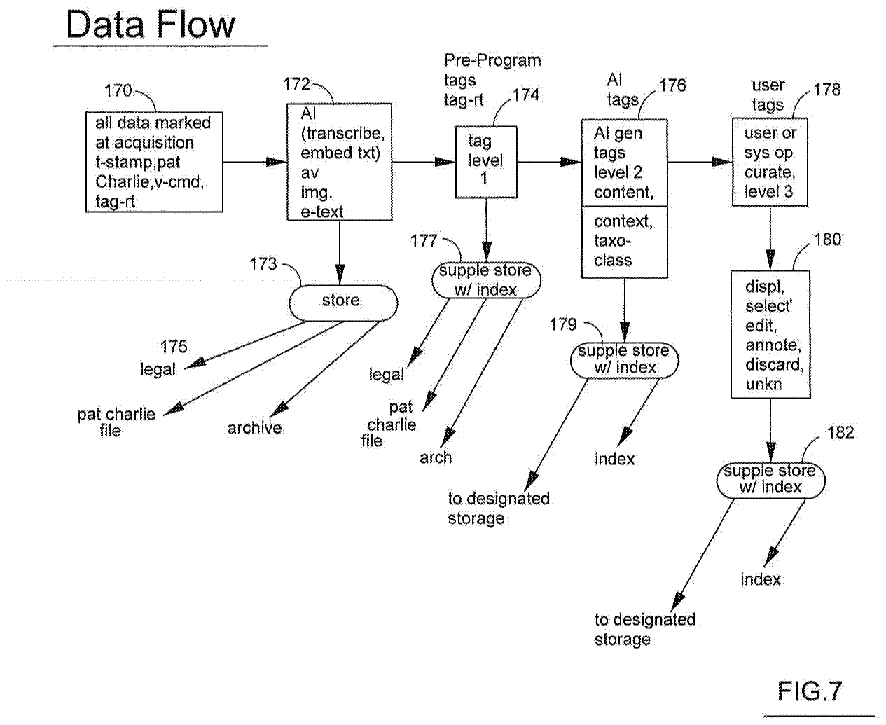

FIG. 7 generally shows data flow for the system. In the first functional block 170, all the data that is acquired is marked in some manner at acquisition. These markings typically include a running timestamp, a device tag (to identify the acquisition device), a patient identifying stamp or tag, any voice commands identified by the VOD-VID systems and any real time tags triggered by the sensed events by the VOD-VID. The time stamp is a date plus time data point. The real time tag tag-rt can be a data point or tag based upon a sensed event such as a v-cmd, med machine ON-OFF, operator manual switch, photo camera flash, audible noise louder than a threshold or a repetitive low volume noise (soft breathing). The next functional block 172 activates the AI process and includes transcribing the voice track and embedding the electronic text E-TXT with the AV/Img to match the acquired voice track data. The AI adds tags for relevance, content, context, and taxo-classes. It should be noted that scribes for an HC provider generate E-TXT based upon physician's dictation (with time stamp tags and commands from the provider). Therefore, AV materials have an embedded or relationally associated E-TXT and images may also have related E-TXT. Of course, manually entered electronic text is also available for processing by the AI process.

Outputs from the AI process stores the material (function fnc 173) into the legal data storage location, the patient Charlie file and an archive data storage or data stores 175. Following the AI process is a function 174 called Tag Level I. This function adds tags to the AV/Img based upon real time tags captured at the data acquisition time. These are pre-AI generated tag-rt. One output from Tag Level I module provides for supplemental data storage fnc 177 and/or data base indexing for legal, patient Charlie PPI file and an archive storage.

After Tag Level I, the AI generated Tag Level II function 176 is activated. This Tag Level II involves adding the content, context and taxonomic functional classification tags discussed earlier at fnc 66, 66a. One AI output is to supplemental data storage fnc 179 with database indexing to the designated storage locations in the database and labels and markers to index the data collections.

The last major function 178 is the application of user generated tags at Tag Level III (tag-u). These user generated tags may be applied by the user engaged in curating the materials or by the system operator. For example, tags-u may be data markers for gen-pop items, in-house med education, unique health issues or concerns, Pat Alpha motivation, Pat Alpha warnings, med supplier, or POI (points of interest). All these tags at Levels I-III can be converted into TXT-Str data and visually presented captions for the data blocks or data segments.

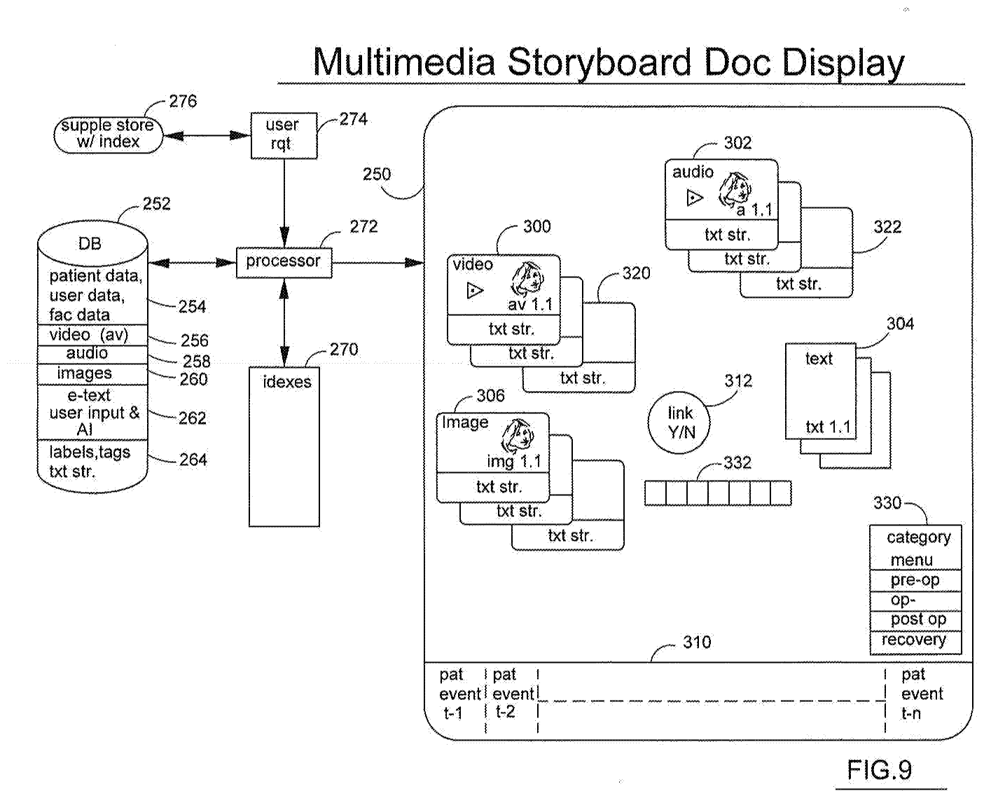

The ultimate output is the permitted display fnc 180 of the TXT-Str marked AI/Img, the selection of AV/Img, the editing of AV/Img, further annotation of AV/Img, potentially discarding the processed AV/Img data block or classifying the processed data block as “unknown.” The unknown category sets aside the AV/Img data block for later consideration and processing. The result from that function 180 is supplemental data storage 182 and indexing to the designated storage location and the index data collection. Key functions are generating time-stamped treatment image data and time-stamped recovery image data such that upon a replay time command from the patient or a healthcare provider, the data block can be displayed using the time-stamped treatment image data as digitally provided captions and time-stamped recovery image data with digitally provided captions. The result, diagrammatically illustrated in FIG. 9 is the substantially simultaneous display of partial views of visual representations of the series of static treatment images, a number of treatment video clips, a series of static recovery images, and a number of recovery video clips, along with a substantially simultaneous display of full or partial views of these respective digital tags for these data blocks, representing TXT-Str for the static treatment images, treatment video clips, static recovery images, and recovery video clips. These visual representations of the respective digital tags permit independent selection of, based upon said user interactive display controller, the static treatment images, treatment video clips, static recovery images, and recovery video clips.

FIG. 8A diagrammatically illustrates the patient workflow scheduling and room manager display 200 (a user interface UI or dashboard (panel)). FIG. 8A shows, in a single view, workflow in an office location at the current calendar day and at the current time. The screen's title indicates this context for clarity (example: “Today [Date] at Dr. Kilder Surgery”). Initially, the view is also within the context of the currently logged in HC user, but can easily be changed in scope to reflect the entire HC-FAC location's users. For example, the doctor working with the patient can log into the system and see the doctor-side workflow for each patient. See U.S. Pat. No. 10,089,439 which describes an integrated HC System Agnostic to user's devices, the content therein is incorporated herein by reference thereto. Each patient for the day starts their journey on the left side of the view in the “Upcoming” list. They progress to the right as their visit continues until they reach the “Completed” list upon conclusion of the visit to the practice facility.

In FIG. 8A, the five vertical columns 202a, 202b, 202c, 202d, 202e show the patient flow for various patients in the locations in the facility where those patients are currently subject to medical services. In the far left column, an indication is provided to the HC authorized person viewing the workflow showing which patients (Judy, Gloria, Jane) should be arriving within the preset time frame designated vertically on the workflow display. Times are not shown in FIG. 8A but would be shown on user interface UI 200. On the far right hand or fifth column 202e the workflow process illustrates patients (Sara, Holly) who have completed treatment or who have recently left the facility. The second column 202b from the left shows the patient (Wilma) who just arrived. The third column 202c from the left shows the patient (Fred, Carmine, Esther) in a particular treatment or examination room and the fourth column 202d from the left shows the patient (Ethan) in the process of checking out and making payment for medical services.

FIG. 8A also shows a methodology for managing patient flow, tasks and facility utilization and diagrammatically illustrates a Practice Flow User Interface (UI) 200. In a typical HC practice, scheduling of patient appointments is used to balance resources such as personnel, rooms within the building and equipment. Prior art calendaring systems seek to ensure efficiency for the HC practice but typically do not yield the best experience for the patient as the typical calendar view does not provide the whole picture. The Practice Flow concept in FIG. 8A presents a holistic view of the current day at the practice and is focused on what is happening for each patient now versus the prior art calender which merely shows a day view of timeslots. Therefore, the Practice Flow UI is patient-centric rather than (I) HC provider centric or (ii) HC facility centric. A quick glance at the inventive Practice Flow User Interface (UI) in FIG. 8A can provide the practitioner and staff with an overall understanding of the patients at that office and their needs. Not only can this view be helpful to understand the current patient-centric demands on the practice and staff, but also enables efficient use of time and resources to ensure an excellent experience for the patients entrusting their care to the practice that day and at that time. This patient-centric UI practice flow is not intended to replace the typical prior art calendar system for scheduling appointments, but it will provide a much more holistic representation of the current day at the practice.

FIG. 8A shows a Workflow and Interaction UI 200 for HC patients Judy, Gloria, Jane, Wilma, Fred, Carmine, Esther, Ethan, Sam and Holly. FIG. 8A illustrates a wire frame for the UI. The entire view is within the context of the single HC office location and the current calendar day.

The HC patient tile 204 for Judy is represented by a tile that flows (moves) through the UI screen from left to right during that patient's visit to the practice. Title 204 is moved based upon the HC authorized user control (which may be a mouse or touch screen control) when Judy tile 204 arrives (column 202b), is received in med room (202c), is moved to check out (202d), and then complete 202e. In one embodiment, each tile includes the following: (a) Patient's Name and Photograph (to assist the HC staff in identifying the patient while in the practice for a more personal touch) (Judy's photo in tile 204 9a happy face symbol); (b) The purpose of the patient's visit to the practice (herein “med act”) (the med act can be used for an automation fnc such as notifying HC practice and HC staff members or displaying content on an appropriate enabled device (smart phone, tablet, iPad (Apple™), laptop or desk top carried by the HC staff) or a display monitor VID or an Apple TVs in the HC facility); © The scheduled appointment time (before the patient arrives at the HC med station) or the elapsed time of their visit (after the patient has arrived); (d) The HC Practitioner's name and photo (“Dr. Smiling Face”) for whom the patient is seeing that day.

The UI Flow Chart represents the overview of the practice for the day. All patients scheduled the day should appear on this UI chart; typically starting their patient journey on the left, in the Upcoming column 202a, and ending at the Complete column 202e on the far right.

Each column in the view represents a state of the patient's visit to the HC practice. The “state of the patient” is a time-based patient status condition. The patient state or status changes as the patient is processed and treated at the HC-FAC. The patient, and hence the tile that represents the patient, flows from one column to the next to signify the various stages of their visit to the practice. The authorized HC user may remain on this UI flow process view and the UI display view will be updated in real time to reflect any changes made to the patient's status/state or information. The HC UI user may also interact with this view by dragging a patient's tile from one column to the next to match the status of their visit or by tapping the patient's tile to select from various menu-based options. For example, when Judy arrives, the HC UI user either (I) drags Judy tile 204 to arrived column 202b or (ii) tile 204 in column 202a presents a drop down menu permitting the HC UI user to select the next HC event for Judy tile 204. If the Sys Op wants to permit the AI to control patient tile, the earlier AI System has that functionality.

The scope of the UI Practice Flow display 200 is the current day at the selected practice, the current time of day, and what is appropriate for the currently logged-in HC UI user of the patient flow system and method. Each HC doctor and HC staff has an enabled device with an app which is keyed into the UI Practice Flow Chart of FIG. 8A. See U.S. Pat. No. 10,089,438. For example, if a HC provider is the current user of UI 200, the “Upcoming” list of patients are only those for that HC provider (Dr. Kilder). If needed, the current HC user may choose to expand the scope of the UI to show all upcoming patients to the HC-FAC including those who are arriving to visit other HC providers (for example Dr. Smith) at that HC med office. Dr. Smith's UI Process Flow 200 would initially show Dr. Smith patients (see UI 200, altered for Dr. Smith), but Dr. Smith could switch views to Dr. Kilder at UI 200 or “all doctors.” The “Received” column 202c shows all HC facility rooms and med equipment at the HC practice. All occupants (Fred, Carmine, Esther) of those rooms are shown so as to clearly indicate which rooms/equipment are occupied at the current time.

All patients for other HC providers are shown on an embodiment of UI 200 (but not shown in FIG. 8A), but in a dimmed or shaded view on the UI display presentation for clarity. The HC UI user may switch to the typical prior art calendar view at any time to view the timeslots for one or more days across one or more users.

The Practice Flow UI view 200 is updated in real time so that it always reflects the current patient status of the HC practice as of right now. The HC UI user may tap on or select any “tile” for the (patient or room) to invoke a drop down menu with options relevant to the current time and status of that item. The HC UI user may drag any patient from one column to another to change their status. Doing so may invoke applicable automations and notifications to the relevant HC staff and HC Providers.

The Upcoming Column (Judy, Gloria and Jane) 202a shows all patients scheduled for the current day who are expected to arrive at the HC-FAC or practice, in the order of “next patient” due at the top of the UI column 202, a list based on the current time of day. Patients that are overdue are highlighted for personal contact to determine if they need assistance or wish to reschedule.

The Arrived Column 202b shows all patients (Wilma) that have arrived at the HC practice and are waiting to be escorted to a HC facility room (treatment, consultation, etc.) in the order of the “wait time since arrival” (longest wait at the top of the list). Once a patient arrives and is checked in, a stopwatch (or count up timer, not shown) in the Practice Flow system 200 is started and runs the entire patient visit. Each patient's tile now shows a split of that stopwatch (count up timer) which represents the elapsed time within the current column. This med location timekeeper again provides an at-a-glance utilization time-based factor. Med facility room and equipment utilization can improve by the data acquisition system capturing real time utilization key performance indicator (KPI) data. Once any segment of the stopwatch for that patient (overall or med room segment) reaches a pre-determined threshold, that patient's tile will reflect that they have been waiting longer than normal (highlight display or flashing warning) and should be personally advised as to status of their visit with the practitioner. These times may also be used by the HC practice to improve their overall efficiency by minimizing patient wait times.

The Received Column 202c is separated into discrete blocks, each representing a HC room or resource (exam room 1 (Fred); exam room 2 (empty); surgery (Carmine); xray (empty); recovery (Esther)) within the HC practice. An empty block represents that the room/resource is currently available. An occupied block (Fred, Camine, Esther) represents that the room or resource is currently occupied by the patient shown in the block. Patients can be moved from one room or resource to another by dragging (or using a drop down menu selection) the patient tile between resource blocks, for example, by moving a patient from an exam room to a surgery room. To aid in determining which rooms/resources are occupied, all patients are shown, although those for other practitioners are shown in a dimmed view and cannot be directly moved without suitable HC professional permission. The dimmed view is not shown in FIG. 8A.

The Checking Out column 202d includes those patients (Ethan) who have completed their HC visit with the practice and are ready to settle their account prior to leaving the office. These patients may still be in a room awaiting a portable payment processing terminal or may have returned to a central location such as the front desk for settlement. Front desk staff may particularly find this column useful as it provides easy access to those patients who are ready to check out.

The Complete Column 202e shows all patients (Sara, Holly) who have completed their visit at the HC practice for this day. The most recently completed patients (Sara) are the top of the list. Patient Holly left the HC-FAC before Patient Sara. These patients can easily be accessed again for reasons such as scheduling a follow-up appointment or contacting them for claiming lost items or assuring compliance with drug treatment, wound care or other post-op patient-centric at home care.

The nominal version of the UI display visually segments into three columns the Process Flow, including (a) an arrived column displaying patients who have arrived at the facility, (b) a treatment room and recovery room column displaying patients in respective treatment rooms and the recovery room, and (c) a check-out column for the facility. The processor and data store control the user interface display based upon image data from the sensory subsystem, typically presence data, timing data (for example, patient unique recovery timing data), and time-stamped data (for example, patient unique recovery time-stamped data). Each patient is represented by a respective patient display tile on the UI and the patient display tiles disposed in one of the five columns (in an enhanced version) or in one of the three columns (in a bare bones version). The system visually presents, in the arrived column and the treatment/recovery room column, the corresponding change in patient status substantially concurrently when the first waiting-room patient transitions from the waiting room to the designated treatment room, and the corresponding change in patient status substantially concurrently when the treated patient transitions from the treatment room to the recovery room. The AI system confirms the treatment/recovery room column display upon detecting the presence of waiting patients in the waiting room with all corresponding waiting patient display tiles disposed in that column. Also the system confirms changes in that waiting room and the treatment/recovery room column by detecting respective changes in patient status when corresponding waiting room patients transition from the waiting room to the respective treatment rooms such that the corresponding respective patient display tiles transition from the treatment/recovery room column to the check-out column display. The five-column system operates on the same basis relating to presence data, voice recognition data, facial recognition data, v-cmd, tags-rt, medical equipment activation/deactivation, E-TXT data, etc.

Automation Elements: As the patient flows through the HC practice UI 200, each change in status (also reflected as moving from one “column” to the next) may trigger an automated operation such as, but not limited to, the following: (i) A patient moving from the “Arrived” column into “Exam Room 1” might send a notification to that patient's HC provider that the patient is ready to be seen in exam room 1. (ii) A patient moving from the “Received” column into the “Checking Out” column might notify the front desk staff that the patient is now ready to check out so that any necessary preparations can be made to minimize the patient's wait time. (iii) A patient moving from the “Checking Out” column to the “Complete” column might automatically be send a “Thank You” video from the HC practitioner to the patient's app.

Also, the HC user controlling UI 200 may select a “Notify of Delay” message to be sent, automatically, to the patient's app as a notification (before or after arrival at the HC practice) or the UI may automatically notify the HC staff so that the staff can personally visit/contact the patient to inform them of the delay. The patient has a computer enabled device or smart phone coupled to a telecomm system for this communication link. See FIG. 1, phone 35. The system generates communication notifications to healthcare providers via the processor and data store based upon confirming changes waiting room column and the treatment-recovery room column by detecting respective changes in patient status.

To assure patient flow through the HC-FAC, the UI process flow is integrated with the sensor rich AI system. There are three ways to assure that patients flow as predicated tor directed through the HC-FAC. First, the when a patient moves from one HC space or room, the authorized user of the process flow UI “moves” the patient tile on the UI to another HC space or room. The authorized user of the process flow UI may be an HC manager or the HC staffer physically directing Pat Alpha to the next room. In relation to FIG. 8D, it is important that Esther in the recovery room be escorted to the check-out space or HC room within a certain timeframe. The AI system records that UI patient tile action. The process flow UI initiates a count up clock (in FIG. 8D, recover clock ON). The AI system is pre-programmed for Esther's treatment to monitor the recovery clock. When the recovery time period exceeds a predetermined threshold, the AI systems send out notifications, first to the designated HC staffer associated with Esther, and then escalates the notice communication to other HC staffers and HC management until Esther leaves recovery and is physically moved to check-out, FIG. 8A.

Although these notice automations indicate that the UI process flow comm notices are focused on increasing FAC operations, provisions for HC staff and HC provider reponses may be included in the UI process flows, such as comm replies from the HC staffer “busy now”, “in 5 minutes”, on break” and “15 minute personal time” notices. The AI system may be flexible to adjust the current patient load to other HC staffers and, over super cylce time frames (multiple days o rweeks), re-adhust the patient load to account for feedback from all types of HC providers. This HC provider feedback is discussed above in connection wiht the doctor who needs more time with patients than other doctors.

Second, the AI system may have a series of pre-set comm notices (also, these pre-sets could be altered upon HC provider controls, such as immediately after surgery). An HC manager may set several time-out thresholds for Esther (a) to be moved from the surgical suite, (b) Esther to remain in recovery, and (c) Esther to be physically directed to check-out. In addition to these pre-set time-out functions, the AI system may automatically include notice communications to other HC doctors, such as the anaesthesiologist interacting with Esther in surgery and call that HC professional to recovery when Esther is in that HC room. The AI system may include a feedback loop to confirm the Pat Alpha was physically in the UI designated room or space, either before the UI patient tile action or after the tile action. If the AI sensory system does not note that Pat Alpha is physically in the UI designated space or room, the AI system (a) visually alters the patient tile, (b) communicates with the authorized user of the process flow; and (c) communicates with the HC staffer most recently interacting with Pat Alpha. The AI system escalates these communicative notifications to HC management as the time differential between the UI tile action and the “confirm location” of Pat Alpha increases. In a highly trusted AI sensory rich system, the AI may move the patient tile only when the designated patient is in the next HC room or space associated with the patient's treatment regime

Third, in a highly trusted AI system, once Esther leaves the surgical suite (as detected by the AI sensors in the suite), the AI system has pre-programmed timeframes for (a) Esther to be detected in recovery room (if NO, the process flow UI triggers visual, and then possible audile time-over alarms and also comm notices are issued to HC staff as needed, with a time-based escalation function), (b) once Esther is in recovery (as per sensor conditions), the AI system then times Esther's recovery with similar over-time alarms and comm notice functions if Esther is not detected at check-out, and (c) once Esther is in check-out, the AI system stops all time-based tracking of Esther's location and the HC staffer(s) who interact wiht Esther.

The UI 200 also performs with a Multimedia Integration system and method. Any Related TV displays/videos/images/slide shows are indicated by an icon within the respective “column” in the Practice Flow view. See, for example, FIG. 8A, in the “Arrived” column 202b for patient Wilma, the UI control “vid” image. In one embodiment, an AppleTV™ multimedia system is integrated into in the Practice Flow system 200. Tapping or selecting the AppleTV or monitor icon on the UI control panel 200 can invoke playback controls for that monitor or AppleTV display in the specific HC facility room, as well as permit the HC staff controlling the UI to select the current video or still image content to be shown on that monitor or AppleTV unit. Selecting the TV-monitor icon permits the UI operator to select a control or a video/image from a drop down menu. The UI control may permit selection of video 1, 2 or 3 or play, repeat, sequence through 1, 2, 3 controls.

The Practice Flow concept also includes the ability to automatically control the content shown on monitor or AppleTV displays in various HC facility rooms within the HC practice, based on the patients in those rooms and the purpose of their visit. Examples of this integration include (I) An AppleTV-monitor in the waiting room (where all “Arrived” patients (Wilma) are situated) might normally show general videos for the practice or advertisements for specials for HC products available in general. When a patient is placed in the “Arrived” column/status, the monitor-AppleTV in the waiting room may now include videos or images related specifically to the purpose of that patient's visit. (ii) An AppleTV-monitor in an exam or consultation room may display a “Welcome” slide show with the patient's name and information about the practitioner and procedure for which they are visiting. (iii) An AppleTV-monitor in a recovery room may display helpful videos or other information specific to the procedure that the patient in that room might find useful. Other mulitmedia systems may be used in connection with the UI Practice Flow 200 discussed herein.

FIGS. 8B, C, D, E and F diagrammatically illustrate an automated notification system and process. FIGS. 8B, C, D, E and F each represent an action at substantially the same time, hence, the notification is a communication or “Comm at T1” at time T1. Upcomming panel 202a triggers two events, one event being a 24 hour pre-arrival notice to Patients Judy, Gloria and Jane and a second reminder communication, such as a text message, to Judy, Gloria and Jane at Comm T1 informing them of their respective appointments at the HC-FAC. The communications by the Process Flow system and method to the patients may be automated text messages, calls to a smart phone or land line phone, or emails, or a combination thereof, with or without a live operator interaction with the patient.

FIG. 8C notes that the process flow system generates a communication to the HC staff or provider, labeled as HC-P1 in the Figure. This communication may be to a staffer smart phone, tablet, laptop or other computer enabled device and may be audible, visual or both to inform the staffer that Wilma has arrived at the HC-FAC. The timer clock on Wilma's panel display begins a count up (timer ON) to note the time Wilma is in the waiting room.

FIG. 8D sends communications to HC-P2, HC-P3, Dr.1, HC-P4, and Dr. 2, respectively related to Fred in exam room 1, Carmine in surgery (then calling Dr.1 to the surgical suite), Esther in recovery and her need for assistance by HC-P4 and a follow-up visit by Dr. 2. When Fred appears in the exam room, Fred's “arrival wait time” clock stops (as noted below, this is KPI data). The AI system and method at panel 202c interacts with the room sensors (VID, VOD, equipment condition, manual “I am here” switch condition, etc.) and queries (“Q” for questions) the sensors to determine when HC-P2 arrives in the exam room and leaves the room (this is a KPI). As explained earlier, the AI system monitors when Fred leaves the exam room (this is a KPI). In connection with Carmine in surgery, the AI system monitors when HC-P3 is in the room and when Dr.1 is in the room. While Esther is in recovery, the AI monitors when HC-P4 is in and out of the room, and repeats the communication to HC-P4 periodically (for example, every 5 min), and notes when Dr.2 is in the room.

FIG. 8E notes that a communication is sent to the HC billing section when Ethan is processed to leave the HC-FAC. The AI may be set to communicate with Dr.1 to say goodbye to Ethan before he leaves (an exit greeting).

FIG. 8F for the complete panel 202e sends a communication to Sara and Holly with a survey request, communication for post-op and at-home steps to improve recovery (the note “Comm Repeat” for Holly notes that the AI regenerates a data comm with Holly similar to the comm with Sara). The AI also complies the patient diary data and produces any needed drug or in-home therapy compliance instructions to the patient.

FIG. 9 shows the multimedia documentation prestation 250 or storyboard 250 for another aspect of the present invention. In a sophisticated system, a database 252 would be used to store patient data, user data, and facility data 254. Multiple relational databases and indices may be used. This database 252 also includes stored video blocks or data streams 256, audio/voice or V data 258, image data 260 and electronic text 262 which is user input as well as AI generated E-TXT. Further, the database includes labels, tags and TXT-Str 264 for the curated AV/Img data streams. The relational databases may use indices to point to specific video segments, E-TXT or images. It may be more efficient to use several indices or indexes 270 which carry tags or labels or data markers to identify the certain segments of the stored AV data, the stored IMG data in the stored E-TXT data.

Since all this data (AV, Img, E-TXT) is time stamped, the labels and tags and the TXT-Str data blocks may be utilized as index markers to avoid multiple copies of the AV/Img/E-TXT in the database. In any event, the processor 272 controls access and extraction of data from the database and cooperates with the index data collections. The processor 272 responds to user requests RQT function 274 and further responds to supplemental data storage 276 and indexing based upon user requests or instructions. In any event, the display 250 is shown as the multimedia storyboard which includes video 300, audio 302, textual material 304, image material 306 as well as a timeline bar 310. In the center of the storyboard the user can first select content (video, audio, etc) which is then highlighted and then select link YES/NO 312. The multi-media is a display monitor with a user interactive display controller. The interactive display controller may be a touch screen, as mouse-driven control, or an IR pointer with a IR sensitive screen/display. In a nominal system, the display monitor substantially simultaneously displays partial views of visual representations of (i) the series of static treatment images, (ii) time-predetermined treatment video clips, (iii) the series of static recovery images, and (iv) time-limited or predetermined recovery video clips; and also substantially simultaneously displays full or partial views of respective digital tags for items (i) through (iv).

The LINK control couples or decouples the current multimedia displays to a common time or HC event. If the YES LINK command is activated, after the user selects one of the videos based upon the TXT-Str displayed tag, the system automatically displays any video plus any audio associated with that video data block as well as any image in that data block and any text (E-TXT) associated with that video data block. This is diagrammatically illustrated in FIG. 9 as video AV a1.1; Audio a1.1; Image Img a1.1; and Text TXT a1.1. If the user selects by touchscreen the third level of video, that is, video AV a1.3 at block 320, the audio track a1.3 for that video is played along with the video. If the user touch screen selects audio a1.3 at block 322, the text a1.3 would be activated and fully displayed as scrolling text when audio data block 322 is played.

The user of storyboard or multimedia UI display 250 may, via touch screen interaction (swipe left, then touch to activate display), flip through video av1.1, av1.2, av1.3 to play one or anther of the videos. The same is true of audio tracks Audio a1.1, a1.2, a1.3.

Additionally, the multimedia storyboard 250 may show in a lower segment a timeline bar 310 thereby permitting the doctor or HC provider to select a particular event at time t-1, t-2 or t-n. These times t-1, t-2, t-3 relate to, for example, pre-op events, examination events, treatment events, post-op events and recovery events in chronologic order. The multimedia storyboard 250 would also have a category menu 330, which could be drop-down menu or top level view with drop down sub-menus from each top level, such that the user could select pre-op data, operation or treatment data, post-op data or recovery data. The horizontal bar display 332 adjacent the third level of the image block 306 may be multiple picture images selectable and expandable to a “full view” by touch by the user.

If the LINK fnc 312 is turned OFF, then the user may show and select a video data stream 300, 320 that is different in time than the text 304 or the displayed image 306. Further, the multimedia storyboard 250 upon selection of the LINK OFF command, may permit the user to link “play video av1.1” with display text txt1.1 and have the image 306 show a different event (Img 1.3) in the patient data record. Stated otherwise, AV a1.1 is displayed concurrently with Text a1.1 but the image is the third level image Img a1.3, thereby permitting the user to see AVa1.1 the treatment or the operation concurrently with a pre-op image Img a1.3 of the patient. Further, the user could read the text a.1.1 while viewing the video.

The LINK function 312 (fnc 312) operates (I) ON to either link the displayed video 300 or 320 to the related text 304 and/or the related image 306 or (ii) OFF to permit disparate presentations of the video, the textual material and/or the static image. The LINK function may have a drop down menu that forces a concurrent play with selected media. Stated otherwise, the LINK ON fnc 312 causes a substantially simultaneous display and presentation of three reproductions of the event, av 1.3, Txt 1.3 and Img 1.3 or a partial “play/display” control. The text string TXT Str labels or captions on the video and on the image assist in identifying related items (av 1.3, Txt 1.3, Img 1.3) on storyboard display 250. This linked presentation of text, images and video assists in an understanding of the procedure and the favorable outcome or assists in identifying why an undesirable outcome was a result of the medical procedure. When link fnc 312 is OFF, the storyboard operator can flip through the videos, the textual material and/or the images at will.

A further enhancement permits the storyboard operator to show sequential images at sequence display block 332. The sequential images in display block 332 may be a series of still or static images taken during surgery (in that sense caption labels TXT Str would all bear a label such as “surgical Img 1,” then Img 2, Img 3, etc.). This static but sequential presentation shows the surgical process.

Another potential function of LINK 312 ON permits the operator to select an event on the time bar 310 ad then automatically display associated media. For example, selection of Pat Event t-2 would automatically call up video av 2.1, text Txt 2.1, and image Img 2.1, which are all timebased linked or associated with Pat Event t-2. Another enhancement to the storyboard would permit the operator to “touch and hold” the link fnc control 312 and generate a dropdown menu permitting the operator to link only selected one of the video, audio, image and textual material.

Timebar 310 shown at the bottom panel of multimedia display board 250 has regions visually marked with well known medical event stages, that is, patient at HC-FAC arrival, patient exam data, patient diagnosis data, patient treatment, patient post-op, patient recovery (day 1, day 2, 3), at-home recovery (day 1, day 2, 3) and compliance. By scrolling through the timeline bar 310, the storyboard user can trigger a related display of the video, the image, the textual material, etc. on the main display portion of the storyboard. It should be noted that the location of these display elements on the storyboard 250 in FIG. 9 is only illustrative. Other positional locations for video, images, textual material, and image sequences can be used.

FIG. 10 shows key performance indicator or KPI process and methodology 400 for the HC operation. Inputs 412, 412a are fed into the KPI processor as shown earlier and discussed in connection with FIG. 3 (see inputs 52, 71a, 71b). The AI (fnc 414) processes the text TXT as well as the voice commands to process the AV and the Img data streams. The time-based TXT result is fed to the KPI engine.

KPIs are healthcare metrics measuring a wide range of conditions and results of a HC operation. A short list of some HC KPIs follow: Patient Wait Time; Average Number Of Patient Rooms In Use At One Time; Staff-To-Patient Ratio; Bed Or Room Turnover; Communication Between Primary Care Physician, Proceduralist, & Patient; Finance; Average Insurance Claim Processing Time & Cost; Claims Denial Rate; Average Treatment Charge; Permanent Employee Wages; Communications; Number Of Media Mentions; Overall Patient Satisfaction; Percentage of Patients Who Found Paperwork To Be “Clearly Written & Straightforward”; Trainings Per Department; Number Of Mistake Events; Patient Confidentiality; Number OfPartnerships With Advocacy Groups; Patient Wait Times By Process Step; Time Between Symptom Onset & Hospitalization; Number Of Visitors (Patients) Who Leave Without Being Seen; Medication Errors; Patient vs. Staff Ratio; and Patient Follow-Up. There are innumerable HC KPIs that have a bearing on the patient, the HC provider, and the HC-FAC. FIG. 10 selects a small sampling of KPIs that effect drug vendors 422, supply vendors 424, human resources 426 at the HC-FAC, and factors related to the FAC operations 428.

Most importantly, the KPIs are customarily linked to patient location and time periods in the HC-FAC, the HC staff/provider versus patient interaction time, med equipment and supply utilization and patient result and follow-up. In this simplistic presentation of a KPI system 400, drug and med supply vendors interactions, HR and FAC operations are tracked with KPIs. The patient-time-room utilization data, the patient-time-staffutilization data, med equipment usage, documentation of treatment and result and follow-up are generally described above in connection with FIGS. 1, 3 and 5A. In FIG. 5A, rather than focus the curation of the raw data on diagnosis, pre-op matters, treatment, post-op, recovery and in-home compliance, the focus of the System Operator (Sys Op) can be to develop KPI data. As an example and in connection with FIG. 1, KPI data is patient time in wait room 20, patient time and staff interaction in the med prep room 24 (including utilization of equipment, supplies and medications therein), patient time and staff interaction in the med exam room 26 (plus equipment, supplies, and drugs), patient time and staff interaction in the treatment room 30 (plus equipment, supplies, and drugs), patient time and staff interaction in the recovery room 32 (plus equipment, supplies, and drugs), and post-visit processes 38, 40 and 41.

Since the holistic AI process captures time based data, movement and interactions with the patient and the staff and HC provider, the use of voice commands v-cmds can document time and events and consumables in the database. Hence, the description and process flow for the patient diary compilation in FIG. 5A can be used as a roadmap to capture KPIs for rooms 20, 24, 26, 30, 32 and the out-of-office or in-home events 38, 40 and 41.

Inputs 412, 412a are fed into the KPI-AI processor 414 as generally discussed earlier in connection with FIG. 3. The AI processes the text TXT in functions 64, 66, 66a, as well as the voice commands to process the AV and the Img data streams and link those data streams or data blocks to the ESI textual data entered by the HC staff via laptop or tablet computers in rooms 24, 26, 30 and 32. The resultant data, which is time stamped, is fed to the KPI engine 416. The user curates the KPI in function 418 with categories (drugs, med supplies, HR staff, and HC-FAC operations) and time based ratios and consumable-based ratios as needed. The AI engine may automatically curate the KPI data based upon data tags. The KPI outputs 420 are then generated by the KPI engine as further curated by the user. The user typically does not generate the KPI data but only selects the categories and the subcategories to generate the ratios indicative of the key performance indicators.

Key performance indicators KPIs can be broken into several different superclasses and several sub classifications. Superclasses include staff, doctors, drug and supply utilization, facility utilization, recovery, and compliance. In connection with staff utilization, the subclasses or KPIs may include HC staff/provider time in each facility room, total patient interaction time by that particular HC staff/provider, number of patients per HC staff/provider per hour or per day or per month, HC staff/provider time with Dr. Kilder, and HC staff/provider time per patient. A doctor KPI subclass may include time per patient, and number of patients per hour, per day or per month. The drug and supply utilization subclasses may include dosage, unitized dosage per patient per day-month-calendar quarter, use of specially treated bandages, and liquid utilization. Facility subclasses for KPIs may include on a per patient basis, wait room occupancy (time per patient, number of patients per time block, per day, per month), wait room time per patient, occupancy ratios for medical preparation area, occupancy ratios for exam room, occupancy ratios for treatment room, and occupancy ratios for recovery room. Subclasses for recovery and compliance KPIs may include drug utilization per patient/facility/HC staff/Dr and time for recovery per patient/facility/HC staff/Dr. Other superclasses and subclasses for KPIs may be developed by this AI data collection processes described herein or known in the HC industry.

In a nominal format, the processor with the data store, generates a plurality of key performance indicators (KPIs) over a business day for the facility based upon: a sum of all patients at the facility that day, patient unique treatment timing data based upon a patient unique waiting room presence sensory data for the respective patients, a sum of all patient treatment timing data for all patients, the patient recovery timing data based upon a patient unique recovery room presence sensory data for respective patients, a sum of all patient recovery timing data for all patients, the number of treatment rooms at the facility, an interaction time between respective patients and corresponding healthcare providers, and the number of healthcare providers at the facility.

Returning to FIG. 10, the KPI outputs 4220 are supplied to drug vendors 422, supply vendors 424, human resources HR 426, and facility operators 428. In connection with drug vendors and supply vendors, that KPI information, is scrubbed of all PII or patient identifying information, other than the facility or part medical staff data, and is supplied to the vendor computer system 430 for utilization review. Also at the vendor station, the vendor may assign a discount factor and/or a loyalty program (function 432) based upon KPI utilization data from a certain HC-FAC, or a group of HC providers or associated HC clinics. The output from vendor functions 430, 432 is supplied to the medical facility operator at the HC-FAC and more importantly to the facility's computer system 434 and the HC-FAC data is updated.

As an example, FIG. 10 illustrates three potential process outputs utilizing KPIs directly related to cost factors. One KPI process output relates to the patient interaction with the medical facility (fnc 436, 438, 440, 442). The second KPI process output relates to the operation the facility by the staff and the doctors (fnc 446, 447). The third KPI process output relates to the billing system for that medical facility (fnc 448).

The first illustrated KPI process in FIG. 10 relates to patient-centric matters and, more particularly, to volume drug discounts or rebates based on usage at the HC-FAC. The local processor 506 and database 516 (FIG. 11) is updated in function fnc 434 of FIG. 10 which generates data on drug usage by patients subject to a rebate plan. As explained later, processor and data store 506, 516 may be configured as a cloud-based processor 506a with an associated cloud-based memory in FIG. 11. In function 416, each patient utilizing the rebated drug has a patient record which is updated. This patient record has a particular rebated drug KPI which is updated because the HC medical facility can relate the rebate discount to a patient and develop loyalty program based on KPI data sponsored by the drug vendor (fnc 422) to a particular patient or group of patients. Therefore, the patient record is updated (fnc 436) either for a discount and/or for a loyalty program unique to the medical center. Functional block 438 discounts the patient's bill for the current or the next-following medical procedure. Functional block 440 issues medical facility loyalty program points to the patient. These items are stored in the patient record. Functional block 442 notifies the patient of the future discount, currently applied drug rebate and/or the loyalty or reward point program increment for the patient and the associated treatment at the medical facility. Drug rebates by the HC-FAC may be processed differently than discussed above.

Regarding the HC staff and doctor/providers at the medical facility and in connection with function fnc 446, the discount and loyalty program by the drug vendor KPI data (or other HC vendors of drugs, equipment usage, supplies, etc. which generate cost reduction/rebate/discounts as trackable KPIs) is also sent to the human resources HR department 426. This HC personnel KPI tracking function 446 is used to reward HC staff and providers, resulting in increased pay, profit-sharing, or in-house loyalty/reward program to the HC staff or the doctor. It should be noted that the HR fnc 446 may be executed after or concurrent with the patient-centric process flow 436, 438, 440 and 442. The HC staff and/or provider is notified of the reward based upon the med supplier rebate program in fnc 446. Lastly in fnc 448, the billing department at the medical facility notes any discount for the drugs or the supply.

It is possible to generate KPIs with the AI process of FIG. 3. The earlier discussion of using an AI process to shorten med treatment times over lunch break hours of 11:00 AM to 2:00 PM is an example of generating a KPI for room assignments and a KPI for HC staff usage without compromising patient expectations and treatment plans. The earlier example also discussed rewarding the patients with discounted medical fees for the patient's efficient use of HC-FAC and HC staff.

At system initialization, the process flow in FIG. 8A schedules patients p1, p2, p3 in, for example, sequential 20 minute time blocks for the same med treatment (p1 at 11 AM, p2 at 11:20 AM; p3 at 11:40 AM). Assuming p1, p2, p3 are diligent over several treatment periods, for example weekly med treatments at HC-FAC, and assuming the AI data acquisition in FIGS. 1 and 3 captures data that p1, p2, p3 are moving through the med room quicker than the 20 minute initial system setting, the AI process 414 and KPI generator 416 (FIG. 10) may suggest to the HC curator in function 418 that there is a relationship between the professions or work habits of p1, p2, p3 and their “less than 20 minute treatment cycles” over the 3 or more weekly treatment super cycles (a super cycle being a time period longer than constituent shorter treatment time period(s)).

More precisely, the AI data acquisition in rooms 24, 26, 30 and 32 (FIG. 1) capture data points at: entry of Pat Alpha, entry of HC staff/provider, v-cmd of events in med room, activation of med room equipment, drug usage (via v-cmd or scan (such as an SKU scan of the package, tube or box)), treatment result for Pat Alpha (v-cmd, image capture, flash detection of camera, manual switch activation by personnel, etc.). This med room data block is uniquely related to respective med rooms 24, 26, 30 and 32 (FIG. 1).

AI function 414 and KPI generator 416 may automatically generate a suggested KPI to the HC curator in func 416 to reduce the “time for treatment” of p1, p2, p3 in those med rooms by relationally linking the professions of p1, p2, p3 to historic “time for treatment” data over the super cycle 3 week time of p1, p2, p3 at the HC-FAC. The KPI generator may suggest that p1, p2, p3 treatment times be reduced to 15 minute time blocks. Ultimately, the HC curator or System Operator (Sys Op) must decide whether a suggested AI generated KPI enhancement is ethical and more beneficial to the patient and the HC staff/provider than the initial HC-FAC utilization settings.

The KPI generator 416 can be trained in a similar manner to the AI system training as described above in connection with FIG. 3. For example, v-cmd KPI GENERATOR PROGRAM ON, triggers a voice-to-text function in the AI system 414 and generator 416. The curator states v-cmd COMPARE TREATMENT ROOM 30b UTILIZATION TO STAFF TIME NOVEMBER 5 and the AI system 414: (I) gathers data for room 30b for the previous year November 5 and generates a list of HC staff/providers active in room 30b; (ii) generates a staff time/patient visit/staffer for room 30b; and (iii) room utilization factor or ratio of total patient time versus total HC-FAC operational time for November 5. Earlier, the AI system 414 is trained to recognize the words: compare, treatment room [id room data], utilization, to [compare and to being contextually linked by the v-cmd], staff time, and November [indicates a time block, followed by a date code].

FIG. 11 diagrammatically shows typical hardware that is earlier discussed in connection with FIG. 1. The voice recording device VOD or the video recording device the VID is placed at several locations throughout the medical facility. For example, a VOD-VID is placed in the waiting room. This device or devices maybe hardwired through an input-output module to a central processor server. As discussed later, the central processor may be a cloud-based processor. If a cloud-based processor is utilized, the local processor at the medical facility uses an input-output device that sends data back and forth through the telecommunications system to the cloud-based processor. The cloud-based processor will also have a database and a data store or collection facility unique to the medical facility. At the medical facility itself, a smaller database and memory store would be typically provided. This database is accessed and processed according to the central processor at the medical facility.

Typically, the VOD-VID communicates via a wireless communication network to the central processor at the medical facility. This is known to persons of ordinary skill in the art. The wireless network uses a router which operates in connection with the input-output I/O device connected to the processor in the medical facility. The VOD-VID operates in the waiting room, a separate device operates in the preparation room, a third device operates in the examination room, a fourth device operates in the treatment room, and a fifth device operates in the recovery room. All of these devices are connected via the wireless communications network or are hardwired via cable to the I/O device coupled to the central processor in the medical facility.

Periodically and sometimes relatively instantaneously, the central processor may activate the telecommunications system I/O device (which may be a router) to communicate with the patient cellular phone. In FIG. 11, Pat Alpha cellular phone interacts with the telephone communication system to upload recovery data videos to the HC-FAC and to download patient diary data from the medical facility processor and database. Further, the vendor system utilizes the telecommunications system and the external I/O to communicate data with the central processor and the local database. The supplier system also uses the telecommunications system to upload data and receive data from the central processor in the medical facility. The telecommunications system typically is called the Internet.

In the drawings, and sometimes in the specification, reference is made to certain abbreviations. The following Abbreviations Table provides a correspondence between the abbreviations and the item or feature.

Abbreviations Table

| | |
|---|---|
| Admin | Administration or Administrator of a system or process |
| add | address - typically in the patient PPI |
| AI | artificial intelligence software or process, voice decoder, OCR, image recognition, |
| | possibly neural network, etc |
| Alb | album - a collection of data blocks |
| alt. | alternate or optional path or step, see also Opt. for optional |
| API | application program interface |
| App | sometimes app - a small program that calls up another program, directed to a server |
| appl. | application |
| appt | appointment |
| arch | archive, as in archived data |
| ASP | application service provider - server on a network |
| auto | automatically |
| av | audio-visual or audio PLUS video, also includes a video without sound or audio track |
| av-txt | an av data set or av stream with the audio segment converted into recognizable text |
| | (OCR text) and embedded or associated with the video segment |
| bd | board |
| cmd | command |
| comm | communications, typically telecommunications |
| comp | computer having an enabled telecomm - communications module |
| CPU | central processing unit |
| cr.cd. | credit card |
| DB | database |
| dele | delete |
| displ | display, typically display an interactive page or display screen |
| doc | document |
| drv | drive, e.g., computer hard drive |
| Dr | doctor, also another Health Care Professional |
| dyn | dynamic |
| Dyn Img Alb | dynamic image album |
| e | encryption |
| educ | education |
| e.g. | for example |
| em | email |
| embed | integrated into the image or video, such a e-text shown with the image or video which |
| | represents audio track, sometimes a textual string of data displayed with the image or |
| | video, sometimes e-text is indexed such that the e-text is displayed when the image or |
| | video is displayed |
| e-text | sometimes E-TXT, electronic textual material, sometimes input by user, sometimes |
| | embedded or associated (maybe via an index whereby the test is displayed concurrently |
| | with the video) |
| equip | equipment |
| Fac | Facility, as in a hospital or any other type of healthcare facility such as a clinic, doctor's |
| | office, surgical center, etc., such as HC-FAC |
| fnc | function, for example, a “save doc” function |
| geo | geographic location or code (geo.loc. is GPS data) |
| gen | generate, create or develop |
| Gen Pop | general population, or a sub-group within the population, such as women who desire |
| | plastic surgery procedures |
| GPS | geo positioning system and location (optionally time data) |
| HC | healthcare |
| HC-P | healthcare provider |
| hist | historic, as in historic data |
| h-link | hyper link to a certain webpage or landing page |
| I/O | input/output |
| IOS | an Operating System typically for Apple (tm) products |
| id | identify |
| IE | Internet-enabled device, like a smart phone, tablet computer, computer, etc. |
| img | image, sometimes Img, such as an electronically stored photo |
| IP add. | internet protocol address of internet enabled device |
| loc | location |
| mat'ls | materials, such a marketing materials to promote product or HCservice |
| med | medical |
| med educ | materials used in connection with medical educational programs |
| mem | memory |
| mess | message as in SMS or text message |
| mic | microphone or audio pickup device |
| mkting | marketing, such as marketing materials |
| ntwk | network, namely a telecomm network, internet, lan local area network |
| obj | object, for example, a data object |
| opt | optional or alternative program or module |
| OS | Operating system for a computer-based or processor based device |
| pat | patient, a particular patient being Pat Alpha, Pat Bravo, Pat Charlie |
| pat-centric | system or process that centers patient, rather than the HC provider |
| pg. | page, typically a web page, may be a landing web page |
| pgm | program |
| ph | phone, namely an internet enabled phone, such as a smart phone |
| ph.no. | phone number |
| POI | point of interest |
| PPI | personal profile information or data, such as data sufficient to identify Pat Alpha, etc. |
| pre/post | before or after a defined event or action |
| prn | print, as in print screen function |
| proc | processor, typically a microporcessor |
| pt | point, as in jump point to another portion of the program |
| Pty | party |
| P/W | password |
| pwr | power |
| Q&A | question and answer |
| Q | question or query |
| rcd | database record or to record or save, audio record (a-rcd), voice to text or “V-Text rcd” |
| | and keypad generated text data as E-TXT |
| re | regarding or relating to |
| rel | release |
| RQT | request |
| rev | review |
| rpt | report |
| rt | real time, may include day and time stamp data |
| RX | medical drugs or medical equipment |
| sch | schedule |
| sec | security |
| sel | select |
| sig cond | signal conditioner |
| smart ph | smart phone coupled to the internet via a telecomm |
| sms | text message |
| spkr | speaker or audio announcement device |
| stmt | statement, as an invoice or bank statement, or payment statement |
| store | to record in computer memory, local, LAN connect memory or cloud-based memory |
| supple | add to or supplement some other item, typically supplement a data store in memory |
| Svr | sever, as in web server |
| Sys | system, typically the cloud based computer server network |
| Sys Op | System Operator |
| t | time, t1 is earlier than t2, t3, sometimes T1 |
| tag | to label or attach a meta tag or label to designate data image, e-text or av |
| tag-gen | generate a tag for a data image or stream |
| tag-rt | a tag, automatically created in substantially real time upon the occurrence of a defined |
| | event, such as a med tech initially activating a computing device when Pat Alpha enters |
| | the FAC room, a real-time tag on a video camera when Pat Alpha enters the FAC room |
| tax or taxo | taxonomic, as in one class of persons or items defined in a classification system |
| taxo-class | class of persons or items defined in a classification system |
| tblt | tablet computer |
| telecom | telecommunications system or network |
| t-link | a time linked or time stamp on an image or av, namely a voice command time stamped |
| | near or on an image or av data stream |
| t-stamp | a time stamp on an image, a video data stream or a recorded audio data stream, typically |
| | t-stamped are created automatically |
| txr | transmitter - receiver device, maybe BLUETOOTH (tm), LAN, wireless telecom |
| | network, or radio frequency |
| txt | textual material |
| txt-str | a text string |
| UI | user interface, typically a display and touch screen or a keyboard, keypad |
| unkn | unknown |
| UPP | user's personal profile, for example a patient completes a UPP prior to inputting data |
| | about his or her situation |
| URL | Uniform Resource Locator, x pointer, or other network locator |
| v | voice or audio data stream |
| v-cmd | a voice command, a computer-based system recognizing a defined voice command from |
| | a user |
| VID | a video monitoring system or sub-system, the VID may have a VOD incorporated therein |
| VOD | a voice monitoring system or sub-system |
| v-txt | the textual representation of an voice or audio data stream |
| w/ | with |
| w/in | within |
| w/out | without |
| wrt | with respect to |

It is known that “kanban” is a method of organizing and managing professional services work. It uses “lean” concepts such as limiting work in progress to improve results. A kanban system is means of limiting work-in-progress and signaling when capacity is available to start new work. This is known as a “pull system.” For intangible goods—invisible work performed by knowledge workers—it is necessary to visualize a kanban system using a kanban board. The board can visualize the work, its workflow, the kanban system, plus various business and delivery risks. Kanban facilitates complex handling of the work and allows real control of the workload.

### Description of System and Method Features

The system described above uses multiple wireless connected devices and integrates with Internet-enabled (IE) devices, such as, smart phone, cell phone with an APP (an access point), tablet computer, computer, or other wireless or IE devices. Computer tablets and other electronic devices may be configured in this manner. The patient's cell phone or IE will have an APP or internet portal permits the person to access the system. If the patient communicates with the system in a voice mode, the patient may interact with an interactive voice response system or module, an IVR. The IVR translates the voice into text data. Data is processed via computer systems, over the Internet and/or on a wireless or wired computer network (LAN or WAN), and computer programs, computer modules and information processing systems prove the stated functionality.

It is important to know that the embodiments illustrated herein and described herein below are only examples, and statements made in the specification do not necessarily limit any of the various claimed inventions. Moreover, some statements may apply to some inventive features but not to others.

The present invention could be produced in hardware or software, or in a combination of hardware and software, and these implementations would be known to one of ordinary skill in the art. Although the preferred embodiment is discussed in connection with a single facility (FAC), the system and method may be distributed over a number of facilities because the data processing functions can be both localized, distributed or be centralized. The separate elements, functions and means for performing functions or steps may be arranged in a different order than discussed herein to improve efficiency. Importantly, several data processing facilities can use the system and use a cloud-based processing platform as the central processor. Although each facility has access to its own unique database of segment of a database, the group of facilities can share the central processing platform's functionality. Further, the KPI may be combined to enhance the discounts and loyalty benefits described earlier.

The operations of the described computing system may be one or more programs or program modules contained on a medium for use in the operation or control of the computer as known to one of ordinary skill in the art. Further, the program, or components or modules thereof, may be downloaded from the Internet of otherwise through a computer network or, in a cloud-based system, data is uploaded as described herein and processed in the cloud and downloaded as needed by the system operator or the various users, including the patients.

Those of skill in the art will appreciate that the various illustrative modules, components, engines, and method steps described in connection with the above described figures and the embodiments disclosed herein can often be implemented as electronic hardware, software, firmware or combinations of the foregoing. To clearly illustrate this interchangeability of hardware and software, various illustrative modules and method steps have been described above generally in terms of their functionality. Whether such functionality is implemented as hardware or software depends upon the particular application and design constraints imposed on the overall system. Skilled persons can implement the described functionality in varying ways for each particular application, but such implementation decisions should not be interpreted as causing a departure from the scope of the invention. In addition, the grouping of functions within a module or step is for ease of description. Specific functions can be moved from one module or step to another without departing from the invention.

Additionally, the steps of a method or algorithm and the functionality of a component, engine, or module described in connection with the embodiments disclosed herein can be embodied directly in hardware, in software executed by a processor, or in a combination of the two. Software can reside in computer or controller accessible computer-readable storage media including RAM memory, flash memory, ROM memory, EPROM memory, EEPROM memory, registers, hard disk, a removable disk, a CD-ROM, or any other form of storage medium including a network storage medium. An exemplary storage medium can be coupled to the processor such the processor can read information from, and write information to, the storage medium. In the alternative, the storage medium can be integral to the processor. The processor and the storage medium can also reside in an ASIC.

The detailed embodiments of the present invention are disclosed herein; however, it is to be understood that the disclosed embodiments are merely exemplary of the invention, which can be embodied in various forms. Further, the terms and phrases used herein are not intended to be limiting; but rather, to provide an understandable description of the invention.

The foregoing description and accompanying drawings illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the claims.

1. A computer-based method for data processing in a sensory rich healthcare facility having a plurality of treatment rooms, a recovery room and a waiting room for a plurality of patients seeking and undergoing treatment by a plurality of healthcare providers at the facility comprising:
for each treatment room, said recovery room and said waiting room, providing a sensory subsystem (a) detecting voice or visual presence of treatment patients, recovery patients and waiting patients collectively comprising said plurality of patients at said facility and (b) capturing image data of said treatment patients and recovery patients in respective treatment rooms and recovery room, said image data consisting of a series of static images or video;
providing a computer processor with a data store which are both coupled to said sensory subsystem;
detecting the presence of said waiting patients in said waiting room with said sensory subsystem and monitoring a patient-unique wait time for each said waiting patient with said processor;
detecting a first change in patient status when a first waiting patient transitions from said waiting room to a first treatment room of said plurality of treatment rooms based upon said sensory subsystem detecting the presence of said first waiting patient in said first treatment room, said first waiting patient then defined as a first treatment patient, and generating first treatment patient timing data, and capturing patient unique treatment image data for said first treatment patient;
detecting a second change in patient status when said first treatment patient transitions from said first treatment room to said recovery room based upon said sensory subsystem detecting the presence of said first treatment patient in said recovery room, said first treatment patient then defined as a first recovery patient, and generating first recovery patient timing data, and capturing patient unique recovery image data for said first recovery patient;
for said plurality of patients at said facility, repeating (a) detecting the presence in said waiting room, (b) detecting a first change in patient status, (c) detecting a second change in patient status, and (d) generating corresponding patient unique treatment patient timing data, patient-unique treatment image data, patient unique recovery patient timing data, and patient-unique recovery image data;
storing in said data store, via said processor, patient unique treatment timing data, patient-unique treatment image data, patient unique recovery timing data, patient unique recovery image data;
digitally tagging and segmenting said patient unique treatment image data based upon said patient unique treatment timing data to generate time-stamped treatment image data, and digitally tagging and segmenting said patient unique recovery image data based upon said patient unique recovery timing data to generate time-stamped recovery image data, wherein the time-stamped treatment image data constitutes a series of static treatment images or a predetermined treatment video clip and wherein the time-stamped recovery image data constitutes a series of static recovery images or a predetermined recovery video clip;
storing in said data store, via said processor, said time-stamped treatment image data and said time-stamped recovery image data; and
upon a replay time command from at least one patient of said plurality of patients and at least one healthcare provider of said plurality of healthcare providers, displaying said time-stamped treatment image data and said time-stamped recovery image data.

2. The computer-based method for data processing in the sensory rich healthcare facility as claimed in claim 1 including:
with said processor, generating a plurality of key performance indicators (KPIs) over a business day for said facility based upon: said patient-unique wait time, a sum of all said plurality of patients at the facility, said first treatment patient timing data, a sum of all treatment patient timing data for all said plurality of patients at the facility, said first recovery patient timing data, a sum of all first recovery patient timing data for all said plurality of patients at the facility, said plurality of treatment rooms at the facility, an interaction time between respective ones of said plurality of patients and corresponding ones of said plurality of healthcare providers, and said plurality of healthcare providers at the facility.

3. The computer-based method for data processing in the sensory rich healthcare facility as claimed in claim 1,
wherein said image data includes both said series of static images and said video,
wherein said time-stamped treatment image data includes both said series of static treatment images and a plurality of predetermined treatment video clips from said video,
wherein said time-stamped recovery image data includes both said series of static recovery images and a plurality of predetermined recovery video clips from said video;
providing a display monitor with a user interactive display controller; and
substantially simultaneously displaying partial views of visual representations of (i) said series of static treatment images, (ii) said plurality of predetermined treatment video clips, (iii) said series of static recovery images, and (iv) said plurality of predetermined recovery video clips; and
substantially simultaneously displaying full or partial views of respective digital tags for (i) said series of static treatment images, (ii) said plurality of predetermined treatment video clips, (iii) said series of static recovery images, and (iv) said plurality of predetermined recovery video clips.

4. The computer-based method for data processing in the sensory rich healthcare facility as claimed in claim 3 wherein said full or partial views of visual representations of respective digital tags permits independent selection of, based upon said user interactive display controller, (i) one static treatment image in said series of static treatment images, or (ii) one predetermined treatment video clip in said plurality of predetermined treatment video clips, or (iii) one static recovery image in said series of static recovery images, or (iv) one predetermined recovery video clip in said plurality of predetermined recovery video clips.

5. The computer-based method for data processing in the sensory rich healthcare facility as claimed in claim 1 including
audible voice command processing by said processor and said data store, and
capturing a plurality of audible voice commands via said sensory subsystem.

6. The computer-based method for data processing in the sensory rich healthcare facility as claimed in claim 5 wherein said data store includes a plurality of voice command data which correspond to respective audible voice commands including a realtime tag command and a point of interest command

7. The computer-based method for data processing in the sensory rich healthcare facility as claimed in claim 6 wherein said plurality of voice command data includes names of healthcare providers and names of patients.

8. The computer-based method for data processing in the sensory rich healthcare facility as claimed in claim 1 including:
providing a user interface display visually segmented into at least three columns including (a) an arrived column displaying patients who have arrived at the facility, (b) a treatment rooms and recovery room column displaying patients in respective ones of said plurality of treatment rooms and said recovery room, and (c) a check-out column for said facility, said processor and said data store controlling said user interface display based upon said image data from said sensory subsystem;
each one of said plurality of patients represented by a respective patient display tile on said user interface display, said patient display tiles disposed in one of said five columns;
visually presenting, in the respective arrived column and the treatment rooms and recovery room column, (i) the corresponding first change in patient status substantially concurrently when said first waiting patient transitions from said waiting room to a first treatment room of said plurality of treatment rooms, and (ii) the corresponding second change in patient status substantially concurrently when said first treatment patient transitions from said first treatment room to said recovery room.

9. The computer-based method for data processing in the sensory rich healthcare facility as claimed in claim 8 including:
confirming said second column display upon detecting the presence of said waiting patients in said waiting room with all corresponding waiting patient display tiles disposed in said second column display;
confirming changes in said second and third column displays by detecting respective changes in patient status when corresponding ones of said waiting patients transition from said waiting room to respective treatment rooms such that the corresponding respective patient display tile transitions from said second column display to said third column display.

10. The computer-based method for data processing in the sensory rich healthcare facility as claimed in claim 9 including:
generating communication notifications to one or more of said plurality of healthcare providers with said processor and said data store based upon confirming changes in said second and third column displays by detecting respective changes in patient status.

11. The computer-based method for data processing in the sensory rich healthcare facility as claimed in claim 1 including:
providing a user interface display visually segmented into five columns including (a) a first column displaying patients scheduled to arrive at the facility, (b) a second column displaying patients who have arrived at the facility, (c) a third column displaying patients in respective ones of said plurality of treatment rooms and said recovery room, (d) a fourth column displaying patients ready to check-out of the facility, and (e) a fifth column showing patients who have either left the facility or who are about to leave the facility, said processor and said data store controlling said user interface display based upon said image data from said sensory subsystem;
each one of said plurality of patients represented by a respective patient display tile on said user interface display, said patient display tiles disposed in one of said five columns;
visually presenting, in the respective second and third columns, (i) the corresponding first change in patient status substantially concurrently when said first waiting patient transitions from said waiting room to a first treatment room of said plurality of treatment rooms, and (ii) the corresponding second change in patient status substantially concurrently when said first treatment patient transitions from said first treatment room to said recovery room.

12. A computer-based method for data processing in a healthcare facility having a plurality of treatment rooms, a recovery room and a waiting room for a plurality of patients seeking and undergoing treatment by a plurality of healthcare providers at the facility comprising:
for each room, providing one or another or both of an audio sensor generating audio data for voice recognition and an image sensor generating image data consisting of a series of static images or video, the audio sensors and image sensors generating presence sensory data for said plurality ofpatients in respective rooms;
for each treatment room, providing a treatment image sensor generating treatment image data consisting of a series of static images or video, said treatment image sensor complementary to said image sensors generating presence sensory data;
for said recovery room, providing a recovery image sensor generating recovery image data consisting of a series of static images or video, said recovery image sensor complementary to said image sensors generating presence sensory data;
providing a computer processor with a data store which are coupled to said audio sensors and image sensors and which are receiving said presence sensory data and said treatment image data and said recovery image data;
when patients transition from a respective treatment room to said recovery room, generating patient unique treatment timing data based upon said presence sensory data in said respective treatment room and storing said patient unique treatment timing data in said data store;
capturing said treatment image data while respective patients are in corresponding treatment rooms, capturing said recovery image data while respective patients are in said recovery room and storing respective patient unique treatment image data and patient unique treatment recovery data in said data store;
when patients leave said recovery room, generating patient unique recovery timing data based upon said presence sensory data in said recovery room and storing patient unique recovery timing data in said data store;
digitally tagging and segmenting said patient unique treatment image data based upon said patient unique treatment timing data to generate time-stamped patient unique treatment image data, and digitally tagging and segmenting said patient unique recovery image data based upon said patient unique recovery timing data to generate time-stamped patient unique recovery image data, wherein the time-stamped patient unique treatment image data constitutes a series of static patient unique treatment images or a predetermined patient unique treatment video clip and wherein the time-stamped patient unique recovery image data constitutes a series of static patient unique recovery images or a predetermined patient unique recovery video clip;
storing in said data store, via said processor, said time-stamped patient unique treatment image data and said patient unique time-stamped recovery image data; and
upon a replay time command from at least one patient of said plurality of patients and at least one healthcare provider of said plurality of healthcare providers, displaying said time-stamped patient unique treatment image data and said time-stamped patient unique recovery image data.

13. The computer-based method for data processing in the healthcare facility as claimed in claim 12 including:
with said processor, generating a plurality of key performance indicators (KPIs) over a business day for said facility based upon: a sum of all said plurality of patients at the facility, patient unique treatment timing data based upon a patient unique waiting room presence sensory data for respective patients of said plurality of patients, a sum of all patient treatment timing data for all said plurality of patients, said patient recovery timing data based upon a patient unique recovery room presence sensory data for respective patients of said plurality of patients, a sum of all patient recovery timing data for all said plurality of patients, said plurality of treatment rooms at the facility, an interaction time between respective ones of said plurality of patients and corresponding ones of said plurality of healthcare providers, and said plurality of healthcare providers at the facility.

14. The computer-based method for data processing in the healthcare facility as claimed in claim 12,
wherein said patient unique treatment image data includes both said series of static patient unique images and said video,
wherein said time-stamped patient unique treatment image data includes both said series of static patient unique treatment images and a plurality of predetermined patient unique treatment video clips from said video,
wherein said time-stamped patient unique recovery image data includes both said series of static patient unique recovery images and a plurality of predetermined patient unique recovery video clips from said video;
providing a display monitor with a user interactive display controller; and
substantially simultaneously displaying partial views of visual representations of (i) said series of static patient unique treatment images, (ii) said plurality of predetermined patient unique treatment video clips, (iii) said series of static patient unique recovery images, and (iv) said plurality of predetermined patient unique recovery video clips; and
substantially simultaneously displaying full or partial views of respective digital tags for (i) said series of static patient unique treatment images, (ii) said plurality of predetermined patient unique treatment video clips, (iii) said series of static patient unique recovery images, and (iv) said plurality of predetermined patient unique recovery video clips.

15. The computer-based method for data processing in the healthcare facility as claimed in claim 14 wherein said full or partial views of visual representations of respective digital tags permits independent selection of, based upon said user interactive display controller, (i) one static patient unique treatment image in said series of static patient unique treatment images, or (ii) one predetermined patient unique treatment video clip in said plurality of predetermined patient unique treatment video clips, or (iii) one static patient unique recovery image in said series of static recovery images, or (iv) one predetermined patient unique recovery video clip in said plurality of predetermined patient unique recovery video clips.

16. The computer-based method for data processing in the healthcare facility as claimed in claim 12 including
audible voice command processing by said processor and said data store;
capturing a plurality of audible voice commands via said audio sensors;
wherein said data store includes a plurality of voice command data which correspond to respective audible voice commands including a realtime tag command and a point of interest command.

17. The computer-based method for data processing in the healthcare facility as claimed in claim 12 including:
with respect to each patient of said plurality of patients and with respect to said waiting room, generating a patient unique waiting room presence sensory data;
with respect to each patient of said plurality of patients and with respect to said treatment rooms and recovery room, generating a patient unique treatment room presence sensory data for the respective patient in the corresponding treatment room, and generating a patient unique recovery room presence sensory data;
providing a user interface display visually segmented into at least three columns including (a) an arrived column displaying patients who have arrived at the facility, (b) a treatment rooms and recovery room column displaying patients in respective ones of said plurality of treatment rooms and said recovery room, and (c) a check-out column for said facility, said processor and said data store controlling said user interface display based upon said image data from said sensory subsystem;
each one of said plurality of patients represented by a respective patient display tile on said user interface display, said patient display tiles disposed in one of said five columns;
visually presenting in a substantially concurrent manner, in the respective arrived column and the treatment rooms and recovery room column: (i) a respective patient display tile in the arrived column based upon the corresponding patient unique waiting room presence sensory data, (ii) a respective patient display tile in the treatment rooms and recovery room column based upon the patient unique treatment room presence sensory data for the respective patient in the corresponding treatment room and based upon the patient unique recovery room presence sensory data for the respective patient in the recovery room.

18. The computer-based method for data processing in the healthcare facility as claimed in claim 17 including:
generating independent communication notifications to one or more of said plurality of healthcare providers with said processor and said data store based upon (i) patient unique waiting room presence sensory data, (ii) patient unique treatment room presence sensory data, and (iii) patient unique recovery room presence sensory data.

19. A computer-based method for data processing in a healthcare facility having a plurality of treatment rooms, a recovery room and a waiting room for a plurality of patients seeking and undergoing treatment by a plurality of healthcare providers at the facility comprising:
for each room, providing one or another or both of an audio sensor generating audio data for voice recognition and an image sensor generating image data consisting of a series of static images or video, the audio sensors and image sensors generating presence sensory data for said plurality ofpatients in respective rooms;
for each treatment room, providing a treatment image sensor generating treatment image data consisting of a series of static images or video, said treatment image sensor complementary to said image sensors generating presence sensory data;
for said recovery room, providing a recovery image sensor generating recovery image data consisting of a series of static images or video, said recovery image sensor complementary to said image sensors generating presence sensory data;
providing a computer processor with a data store which are coupled to said audio sensors and image sensors and which are receiving said presence sensory data and said treatment image data and said recovery image data;
providing a user interface display visually segmented into at least three columns including (a) an arrived column displaying patients who have arrived at the facility, (b) a treatment rooms and recovery room column displaying patients in respective ones of said plurality of treatment rooms and said recovery room, and (c) a check-out column for said facility, said processor and said data store controlling said user interface display based upon said image data from said sensory subsystem;
each one of said plurality of patients represented by a respective patient display tile on said user interface display, said patient display tiles disposed in one of said five columns;
when patients transition from the respective treatment room to said recovery room, generating patient unique treatment timing data based upon said presence sensory data in said respective treatment room;
capturing said treatment image data while respective patients are in corresponding treatment rooms, capturing said recovery image data while respective patients are in said recovery room and storing respective patient unique treatment image data and patient unique treatment recovery data in said data store;
visually presenting in a substantially concurrent manner, in the respective arrived column and the treatment rooms and recovery room column, (i) the patient transition from said waiting room to the respective treatment room, and (ii) the patient transition from the respective treatment room to said recovery room;
digitally tagging said patient unique treatment image data and patient unique recovery timing data, wherein said patient unique treatment image data constitutes a series of static patient unique treatment images or a predetermined patient unique treatment video clip and wherein said patient unique recovery image data constitutes a series of static patient unique recovery images or a predetermined patient unique recovery video clip; and
upon a replay time command from at least one patient of said plurality of patients and at least one healthcare provider of said plurality of healthcare providers, displaying said patient unique treatment image data and said patient unique recovery image data.

20. A computer-based method for data processing in a healthcare facility having a plurality of treatment rooms, a recovery room and a waiting room for a plurality of patients seeking and undergoing treatment by a plurality of healthcare providers at the facility comprising:
for each room, providing an audio sensor generating audio data for voice recognition and an image sensor generating image data consisting of a series of static images or video;
generating presence sensory data for said plurality of patients in respective rooms with said audio sensors and image sensors;
for each treatment room, providing a treatment image sensor generating treatment image data consisting of a series of static images or video, said treatment image sensor complementary to said image sensors generating presence sensory data;
for said recovery room, providing a recovery image sensor generating recovery image data consisting of a series of static images or video, said recovery image sensor complementary to said image sensors generating presence sensory data;
providing a computer processor with a data store which are coupled to said audio sensors and image sensors and receiving said presence sensory data and also receiving said treatment image data and said recovery image data;
when patients transition from a respective treatment room to said recovery room, generating and capturing patient unique treatment timing data based upon said presence sensory data in said respective treatment room;
capturing patient unique treatment image data for respective patients who are in corresponding treatment rooms;
capturing patient unique recovery image data for respective patients who are in said recovery room;
when patients leave said recovery room, generating patient unique recovery timing data based upon recovery room presence sensory data;
digitally tagging and segmenting said patient unique treatment image data based upon said patient unique treatment timing data to generate time-stamped patient unique treatment image data;
digitally tagging and segmenting said patient unique recovery image data based upon said patient unique recovery timing data to generate time-stamped patient unique recovery image data;
wherein said time-stamped patient unique treatment image data constitutes a series of static patient unique treatment images or a predetermined patient unique treatment video clip and wherein said time-stamped patient unique recovery image data constitutes a series of static patient unique recovery images or a predetermined patient unique recovery video clip;
storing in said data store, via said processor, said time-stamped patient unique treatment image data and said patient unique time-stamped recovery image data;
providing a display monitor with a user interactive display controller; and
substantially simultaneously displaying partial views of visual representations of medical data consisting of (i) said series of static patient unique treatment images, (ii) said plurality of predetermined patient unique treatment video clips, (iii) said series of static patient unique recovery images, and (iv) said plurality of predetermined patient unique recovery video clips; and
with said partial views of visual representations of medical data, substantially simultaneously displaying full or partial views of respective digital tags for (i) said series of static patient unique treatment images, (ii) said plurality of predetermined patient unique treatment video clips, (iii) said series of static patient unique recovery images, and (iv) said plurality of predetermined patient unique recovery video clips.
